(19) United States
(12) Patent Application Publication
(10) Pub. No.: US 2019/0231927 A1
(43) Pub. Date: Aug. 1, 2019

(54) COMPOSITE-INTERFACING BIOMATERIAL ACCELERANT SUBSTRATE

(71) Applicant: PolarityTE, Inc., Salt Lake City, UT (US)

(72) Inventors: Denver Lough, Salt Lake City, UT (US); Nikolai Sopko, Salt Lake City, UT (US); Pratima Labroo, Salt Lake City, UT (US); Nicholas William Baetz, Draper, UT (US); Jennifer Jensen Irvin, Salt Lake City, UT (US); Georgia Crystal Yalanis, Millcreek, UT (US); Matthew Petney, Salt Lake City, UT (US)

(21) Appl. No.: 16/260,052

(22) Filed: Jan. 28, 2019

Related U.S. Application Data
(60) Provisional application No. 62/622,489, filed on Jan. 26, 2018.
(60) Provisional application No. 62/776,329, filed on Dec. 6, 2018.

(51) Int. Cl. A61L 27/36

(52) U.S. Cl. CPC A61L 27/3604; A61L 27/3687; A61L 2430/00; A61L 27/3608; A61L 27/3612

(57) ABSTRACT

Disclosed herein is a composition comprising stimulated biological material derived from an interface compartment, wherein the composition is capable of augmenting the generation or healing of a native tissue when administered to a subject in need thereof.

### CROSS-REFERENCE TO RELATED APPLICATIONS

This application claims priority to U.S. provisional application No. 62/776,329, filed Dec. 6, 2018, and U.S. provisional application No. 62/622,489, filed Jan. 26, 2018. The contents of both applications are incorporated by reference in their entirety herein.

### TECHNICAL FIELD

The present disclosure relates generally to the development of biomaterial containing composition(s) which alter triploblastic-derived multicellular systems through action on intermediate substrates.

### BACKGROUND

The ability to directly or indirectly impact biological material systems and/or to activate, enhance, and/or modulate functional activity in a target biomaterial has long been a goal of conventional biomedical engineering efforts. Traditional approaches to biomaterials and/or biomedical engineering are often designed around the classically taught tissue engineering triad whereby a cell type, a molecular agent and/or scaffold/matrix are used in singularity or combination to enhance or augment processes in the tissue in which agent is placed. As such, said agents: cellular entities, molecular agents and/or scaffolds/matrices are isolated, synthesized and/or constructed in isolation of more complete systems involving interactome(s), which leads to limits, voids and/or insufficiencies (e.g., cellular senescence, molecular misapplication, adverse microenvironment selection and/or scaffold/matrix artificialization). Such reductionist approaches in the development of biomaterial substrates result in incomplete adynamic systems. Subsequently, such incomplete systems inherently alter the substrates' intrinsic equilibrium and/or impact external systems, resulting in unintended consequences and/or disequilibrium of the entire system.

Commonly, organized biological systems derived from triploblastic origins are developed from three primary germ layers generally referred to as: ectoderm, mesoderm and endoderm. From such germ layers, organized cellular architecture, function and life are generated. Appropriate propagation of such germ layers and the resulting “inter” and “intra” interactions which occur between and/or within such germ layers lead to the formation of more advanced structures (e.g., appendages, tissues, organs).

In triploblastic organisms, the reduction of cellular potency during phased embryonic development and associated propagation of germinal layers occurs, in part, as a result of relative changes to intracellular, intercellular, extracellular, transcellular and/or pericellular interactome(s). Such changes to cellular and/or subcellular organization lead to the progressive formation of more ordered structure(s), more complex substrate(s) and more functional system(s). The relative, progressive and changing orientation as well as physiologic polarity of such entities and/or advanced structures occurs, in part, due to interfaced flux gradients of organic and inorganic agents that are present between activators and responders and thus can act on either and/or both. These agents correlate to discrete cause and effect mechanisms/relationships.

While states of cellular potency and organization of cellular entities and associated material(s) transition and change throughout progressive maturation and development, fundamental elements of such physiology remain conserved. Some of these changes and/or maintained conservation to structural orientation and function relate to effects of interactive agents located between and/or within intracellular, intercellular, extracellular, transcellular, and/or pericellular interactome(s) and the relative interfacing dynamics between such organic and inorganic agents, cellular entities, and/or associated material(s).

Tissue(s), a basic example of a functional organized cellular entities, have organized groups of interacting cells having a common structure and function. Physiologically, mammalian tissues are organized into four basic categories: epithelial (e.g., skin), connective (e.g., loose connective tissue, dense connective tissue, ligaments, tendons, cartilage, and bone), muscular (e.g., cardiac tissue, smooth tissue, and skeletal tissue) and nervous. Each type of tissue plays a unique role in the maintenance of biological life. As such, disruption of tissue can result in injury, disease, or loss of life.

When considering deleterious acts and/or destruction of advanced structures in triploblastic-derived systems (e.g., tissues), generation, regeneration, and/or neo-generation of the tissue structure(s) is preferred to mere reparation of the disrupted structure(s) because reparation can result in inadequate repair of the structure through fibrosis, scar formation and disorganization. Accelerated forms of healing are desired over scar formation because they result in greater functional capabilities of the resulting structures and/or associated systems.

Skin is an exemplary tissue where accelerated forms of healing such as neo-generation and/or regeneration are desirable over scar formation. Skin is a vital and critical organ serving essential needs including physical and mechanical barrier protection, immunologic protection from pathogens, thermoregulation, and somatic sensation, as well as providing exocrine and endocrine roles. The physical and structural integrity of the skin must be maintained in order for the integumentary system to function.

Further examples of the intricacies surrounding critical interdependent elements within the interactome(s) can be observed in cutaneous wound healing which involves a myriad of complex, evolutionarily conserved cascade(s) of intracellular, intercellular, extracellular, transcellular, and pericellular events which is commonly simplified into four basic and conventionally progressive phases: (1) hemostasis; (2) inflammation; (3) proliferation; and (4) maturation. Triploblastic-derived tissue systems, when damaged and/or altered outside of the normal spectrum of fluctuation(s), often respond through phased repair processes. Throughout the progression of these phases, a spectrum of irregularities can become present, in part because of time, space and/or material limits within and/or between the interfacing compartments and/or interactome(s). An inverse relationship exists between such irregularities and the generation, regeneration, and neo-generation of native and/or semi-native structure, function, orientation, processes or downstream states.

An example of limit-correlative irregularities within the integumentary system, which contains skin tissue, can be seen in scar formation. Scar tissue(s) are compositionally and structurally different than normal cutaneous tissue(s). Regarding composition, scar tissue is largely comprised of irregularly orientated extracellular materials, altered relative rations of cellular entities/populations and thus different interfaced gradients and interactome profiles. For example, a reduction in oxygen gradients through cutaneous systems select for cellular populations which can viably function in such setting. In such setting, increased levels of myo-fibroblast populations become present and subsequently contribute to the synthesis and deposition of irregularly oriented extracellular materials. These materials and associated cellular populations further effect the system so as to augment scar formation, contraction, and the higher cross-linked, denser, less elastic collagen structures. Selective pressures, resultant from change(s) in environment, cellular entities, relative gradients, interface agents and/or interactome profile result in furthering the select presence of agents, materials, substrates, products and entities within the system. As these selective pressures further direct select compositions of variables, the entire system reorients and/or redirects elements of the interactome(s) and intracellular, intercellular, extracellular, transcellular, and/or pericellular compartment interfaces.

Associated limits within the field which have prevented such capable technology have stemmed from classic teachings and associated algorithms that focus primarily on three major independent components: enriched stem cell entities, classic fixed growth factors and/or synthesized scaffolds or matrices. While important, such components remain incomplete without consideration of the interface(s) and associated interactome(s) which drive dynamic processes and interactions in such intracellular, intercellular, extracellular, transcellular, and/or pericellular compartment(s).

Biomaterials are substances, agents, and/or components that have been developed, assembled, and/or directed to take a form and/or function which alone or as part of a larger system can be used to control, impact, and/or alter interactions of living and/or dynamic systems. Such biomaterials can be further used to control, impact, and/or alter greater systems, which can later react to downstream effects of such greater systems.

Accelerant(s), as they relate to biomaterials or biological systems and/or subcomponents of such, promote change(s) within said system by driving, augmenting, modulating, altering, and/or otherwise impacting forms of cause and effect relationships.

With such understanding of the value of the discrete selective pressures within the composite interactome and/or intracellular, intercellular and/or extracellular compartment interface(s) in directing the orientation, structure, reactivity, function and/or downstream outcome(s) of biophysically responsive material(s), substance(s) and/or substrate(s), there is a present need for improvements in generation, regeneration, and neo-generation of self-propagating structures.

### SUMMARY

The invention relates generally to a composition of biomaterial accelerant substrates and processes for developing activated biomaterial compositions from multi-cellular systems and the compositions produced therefrom. For convenience the invention will be referred to as a Composite-Interfacing, Biomaterial Accelerant Substrate (CIBAS).

One aspect of the present disclosure relates to the generation, neo-generation, and/or regeneration of organized structures which can include but are not limited to appendages, interfaces, tissues and/or organs and associated sub-components.

Another aspect of the present disclosure relates to utilization of the technology to effect a system in which CIBAS is combined with materials and/or matter through direct or indirect effects which include but are not limited to the activation, enhancement, and/or modulation of the greater system.

Another aspect of the present disclosure relates to the utilization of the technology as a transfer agent for other forms of matter which may include, but are not limited to, the following properties and/or functions: vector, carrier, medium, combined material for transfer and/or storage.

Another aspect of the present disclosure relates to the utilization of the technology as a substrate, input, additive and/or supplement to other materials, entities, systems, formulations and/or forms of matter.

An aspect of the present disclosure relates to a composition comprising stimulated biological material derived from an interface compartment, wherein the composition is capable of augmenting the generation or healing of a native tissue when administered to a subject in need thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 depicts laboratory rat specimens L71 and L72 exhibiting the effect of a composition disclosed herein.

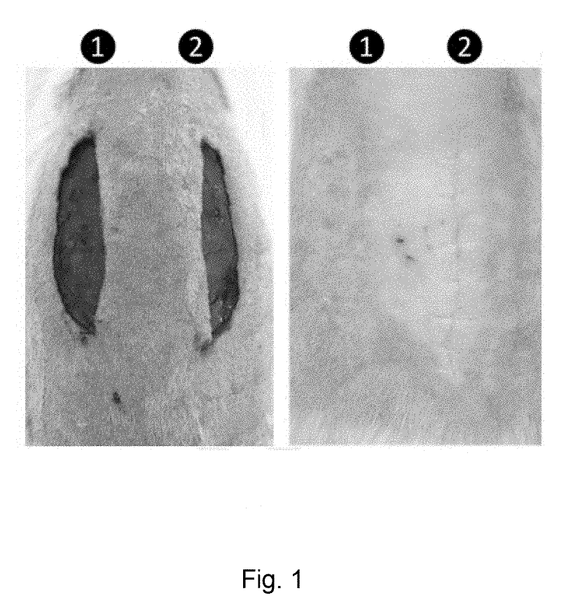

FIG. 2 depicts the average Raman spectra of material prepared from rabbit chondral specimen in Example 5. Average Raman spectra of a rabbit cartilage-derived solution (top) and a rabbit cartilage-derived gel (bottom) are provided.

FIG. 3 depicts the average Raman spectra of material prepared from rabbit osseous specimen in Example 6. Average Raman spectra of a rabbit long bone-derived solution (top), a rabbit long bone-derived freeze-dried gel (middle), and a rabbit long bone-derived gel (bottom) are provided.

FIG. 4 depicts the average Raman spectra of material prepared from rabbit long bone with surrounding muscle specimen in Example 7. Average Raman spectra of a rabbit long bone with surrounding muscle-derived solution (top), a rabbit long bone with surrounding muscle-derived freeze-dried gel (middle), and a rabbit long bone with surrounding muscle-derived gel (bottom) are provided.

FIG. 5 depicts the average Raman spectrum of material prepared from rabbit lumenal osseous (marrow) specimen in Example 8.

FIG. 6 depicts the average Raman spectra of the material prepared from rabbit muscle specimen in Example 9. Average Raman spectra of a rabbit muscle-derived solution (top), a rabbit muscle-derived freeze-dried gel (middle), and a rabbit muscle-derived gel (bottom) are provided.

FIG. 7 depicts the average Raman spectrum of the material prepared from rabbit tendinous connective tissue specimen in Example 10.

FIG. 8 depicts the average Raman spectra of the material prepared from rabbit osseous vertebral specimen in Example 11.

FIG. 9 depicts rheometry data as discussed in Example 12 from rabbit long bone with surrounding muscle-derived gel at shear rates 25.12 1/s (orange), 158.1 1/s (green), and 1000 1/s (blue).

FIG. 10 depicts viscosity vs. temperature of a gel prepared from rabbit muscle as discussed in Example 12 at shear rates 25.12 1/s (orange), 158.1 1/s (green), and 1000 1/s (blue).

FIG. 11 depicts viscosity vs. shear rate of a gel prepared from rabbit vertebrae at pH 6.5 and pH 7.5 as discussed in Example 12.

FIG. 12 depicts the modulus of elasticity (kPA) of certain compositions disclosed herein following cryodesiccation using compression testing. The range of values indicates the difference in strength of the different pore-sized scaffolds.

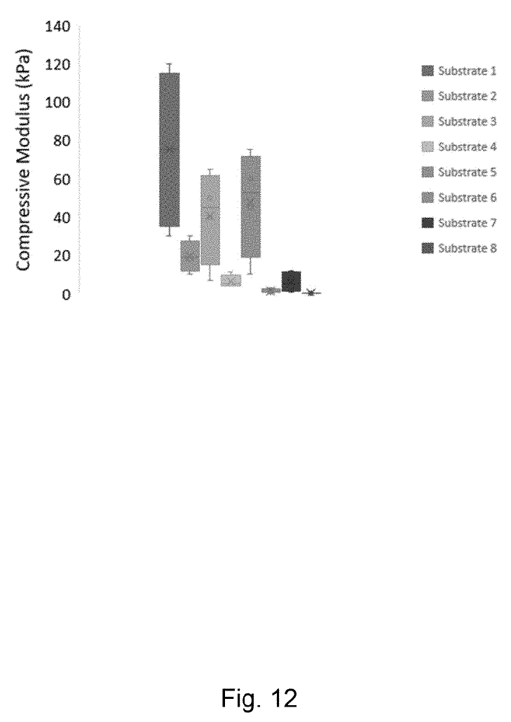

FIG. 13 depicts the modulus of elasticity (kPA) of certain compositions disclosed herein following cryodesiccation using compression testing.

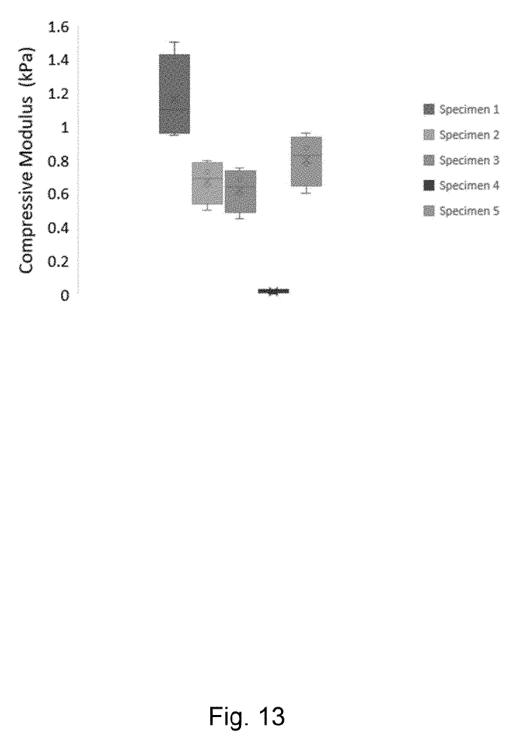

FIG. 14 depicts structural characterization of cryodesiccated osseous-derived compositions disclosed herein: (top) Brighfield microscopic image, (center) Multiphoton confocal image showing structure, and (bottom) Scanning electron microscope (SEM) showing porous structure.

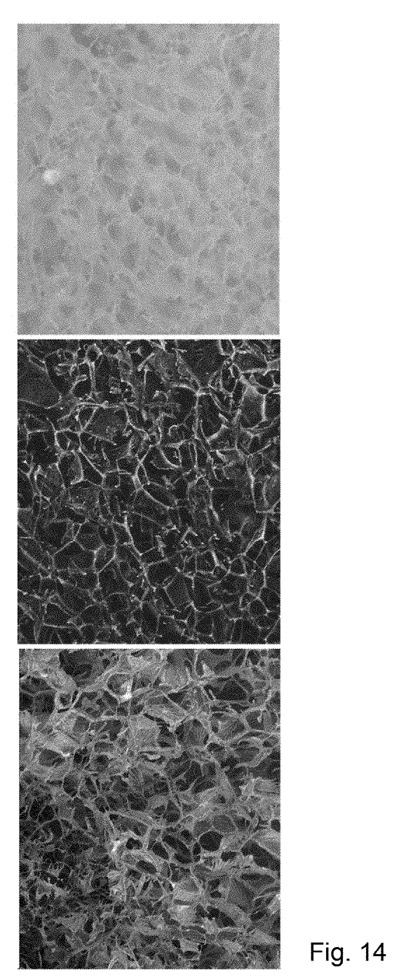

FIG. 15 depicts structural characterization of cryodesiccated myo-derived compositions disclosed herein: (top) Brighfield microscopic image, (center) Multiphoton confocal image showing structure, and (bottom) Scanning electron microscope (SEM) showing porous structure.

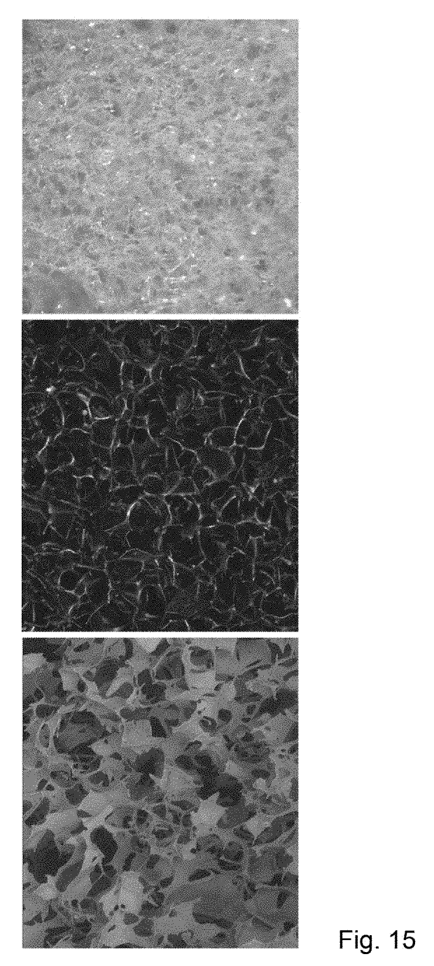

FIG. 16 depicts structural characterization of cryodesiccated chrondral-derived compositions disclosed herein: (top) Brighfield microscopic image, (center) Multiphoton confocal image showing structure, and (bottom) Scanning electron microscope (SEM) showing porous structure.

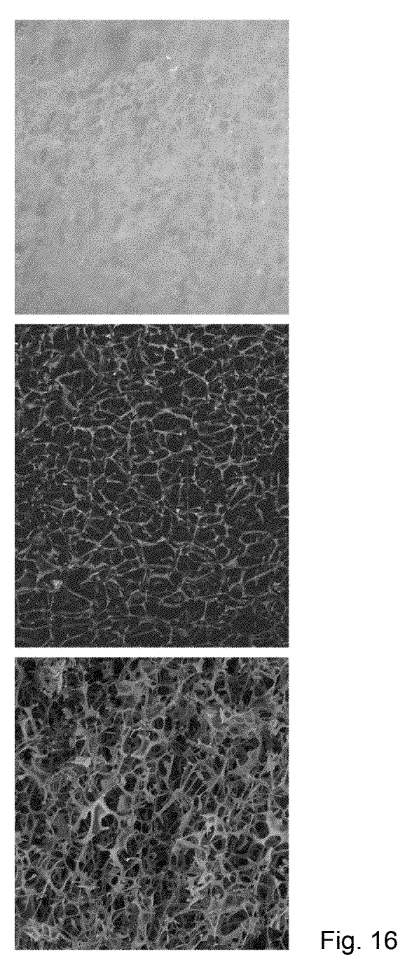

FIG. 17 depicts certain nanoparticle characterization of fractionated fluid compositions disclosed herein. H# indicates fraction with correlative particle profiles and quantity. Such particles are those that exhibit certain brownian motion characteristics.

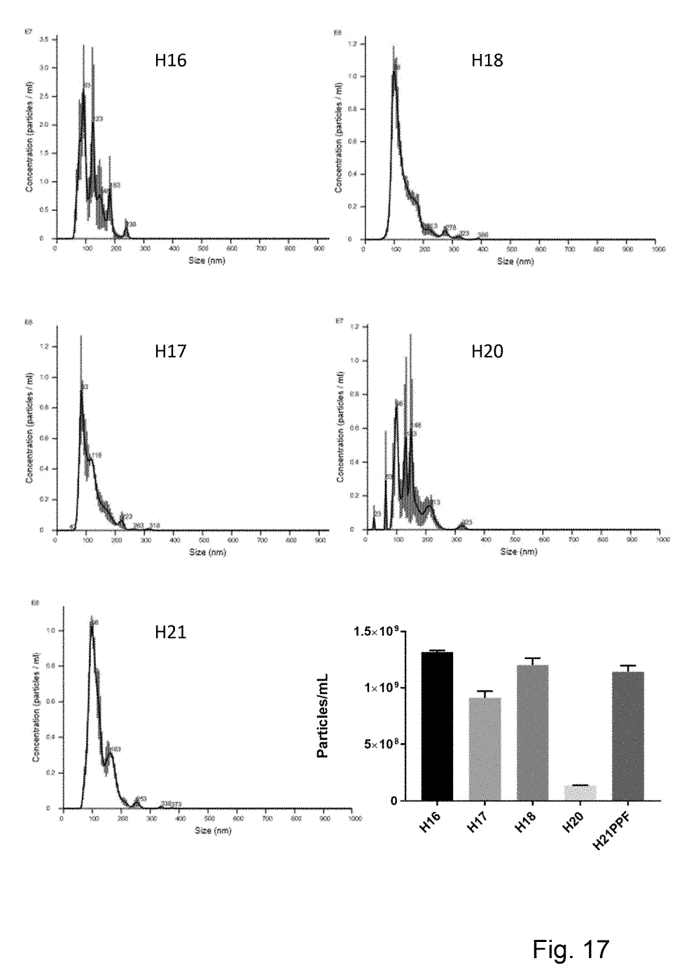

FIG. 18 depicts certain visual characterization of compositions disclosed herein.

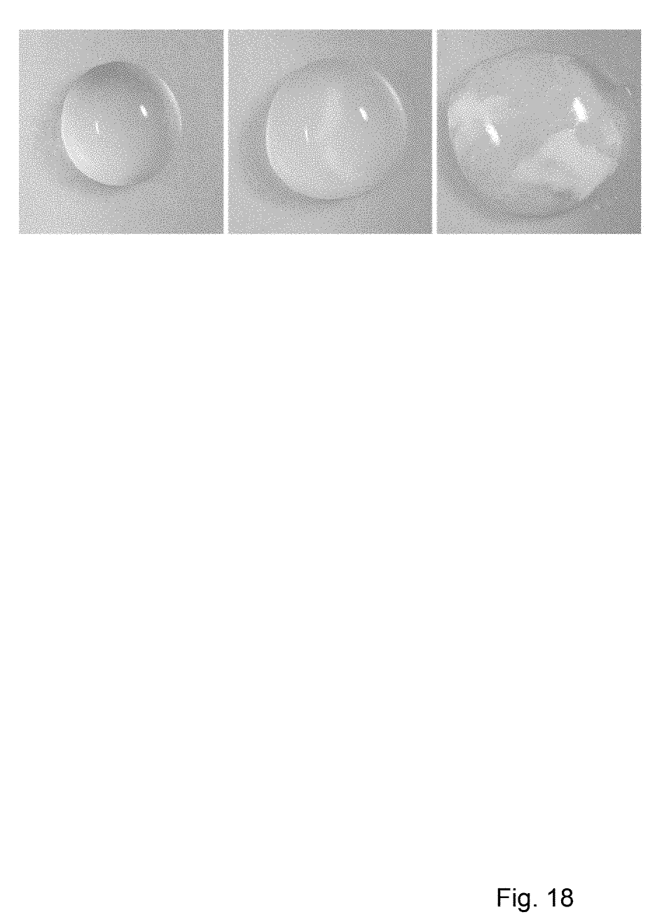

FIG. 19 illustrates various interactomes.

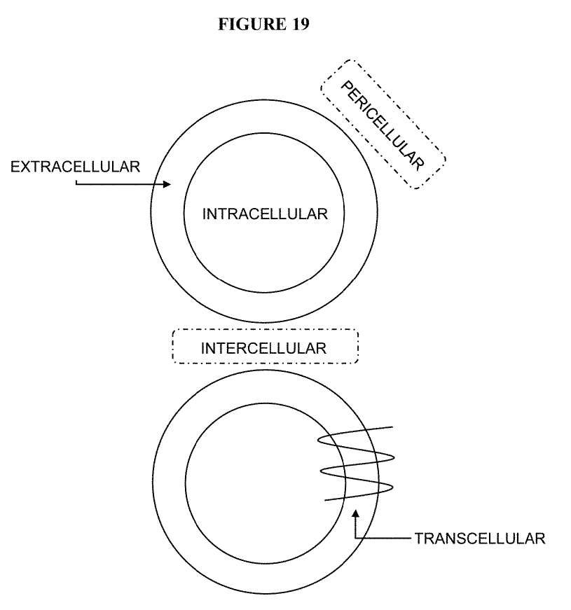

FIG. 20 shows compressive modulus of compositions (e.g., CIBAS) as measured according to Example 15.

FIG. 21 shows protein concentrations for mouse muscle-derived compositions (e.g., CIBAS) as determined according to Example 16.

FIG. 22 shows protein concentrations for rabbit bone-derived compositions (e.g., CIBAS) as determined according to Example 16.

FIG. 23 shows comparative protein concentrations for mouse muscle-derived and mouse bone-derived compositions as determined according to Example 16.

FIG. 24 shows comparative protein concentrations for mouse muscle-derived and mouse bone-derived compositions as determined according to Example 16.

FIG. 25 shows protein concentrations for mouse bone-derived compositions as determined according to Example 16.

FIG. 26 shows concentrations of measured biomarkers for a mouse muscle/bone-derived composition, mouse muscle-derived compositions, and a mouse bone-derived compositions as determined according to Example 17.

FIG. 27 shows concentrations of osteoprotegerin for a mouse muscle/bone-derived composition, mouse muscle-derived compositions, and mouse bone-derived compositions as determined according to Example 17.

FIG. 28 shows concentrations of SOST for a mouse muscle/bone-derived composition, mouse muscle-derived compositions, and mouse bone-derived compositions as determined according to Example 17.

FIG. 29 depicts comparative Raman spectra of a rabbit muscle-derived composition (e.g., CIBAS) (bottom) and native rabbit muscle (top) as measured according to Example 18.

FIG. 30 depicts comparative Raman spectra of a rabbit fat-derived composition (e.g., CIBAS) (bottom) and native rabbit fat (top) as measured according to Example 18.

FIG. 31 depicts comparative Raman spectra of a rabbit cartilage-derived composition (e.g., CIBAS) (bottom) and native rabbit cartilage (top) as measured according to Example 18.

FIG. 32 depicts comparative Raman spectra of a rabbit bone-derived composition (e.g., CIBAS) (bottom) and native rabbit bone (top) as measured according to Example 18.

FIG. 33 depicts comparative Raman spectra of a human skin-derived composition (e.g., CIBAS) (bottom) and native human skin (top) as measured according to Example 18.

FIG. 34 shows results of a cell viability experiment according to Example 23.

FIG. 35 shows concentrations of IL6, osteoprotegerin, and insulin for a liver-derived composition (e.g., CIBAS) as determined according to Example 17.

FIG. 36 shows concentrations of IL6, osteoprotegerin, insulin, and leptin for a cartilage-derived composition (e.g., CIBAS) as determined according to Example 17.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention. Thus, it is to be understood that other embodiments may be utilized and changes may be made without departing from the scope of the present disclosure. The following detailed description, therefore, is not to be taken in a limiting sense.

The present disclosure relates generally to compositions derived from triploblastic multicellular systems in which an interface compartment is disrupted and intracellular, intercellular, extracellular, transcellular and/or peri-cellular interactome(s) therein are combined and thus activated. The present disclosure also relates generally to methods of making such compositions and uses of such compositions.

Aspects of the present disclosure relate to combining the composition with a biocompatible transfer agent for downstream utility.

Aspects of the present disclosure relate to combining the composition with additional materials, composite material(s), and/or matter. Also disclosed herein are combinations of the composition with additional materials, composite material(s), and or matter.

Aspects of the present disclosure relate to a composition that augments, promotes, regulates, and/or inhibits processes utilized in a triploblastic-derived multicellular system.

Aspects of the present disclosure relate to a composition that alters processes involved in anabolism, catabolism and/or metabolism utilized in cellular entities and/or cellular-based systems.

Aspects of the present disclosure relate to a composition that accelerates cellular and/or tissue functional activities.

Aspects of the present disclosure also relate to a composition that prevents or reduces the disorganization of cellular or tissue structures (e.g., included but not limited to cellular senescence, scar formation and fibrotic processes within tissues and multi-cellular systems).

Aspects of the present disclosure relate to selectively capturing and altering pericellular interfaces of triploblastic-derived specimen and activating and isolating a stimulated composition.

Disclosed herein is a composition comprising stimulated biological material derived from an interface compartment. The composition is capable of augmenting the generation or healing of a native tissue when administered to a subject in need thereof.

In an embodiment, the stimulated biological material derived from the interface compartment is acellular. In an embodiment, the stimulated biological material comprises biological material derived from a heterogeneous population of mammalian tissue interface cells. In an embodiment, the stimulated biological material derived from the interface compartment comprises a plurality of interactomes associated with the heterogeneous population of mammalian tissue interface cells.

In an embodiment, the stimulated biological material includes living core potent cellular entities and supportive entities. In an embodiment, the living core potent cellular entities express RNA transcripts and/or polypeptides of one or more Leucine Rich Repeat Containing G Protein-Coupled Receptors selected from the group consisting of LGR4, LGR5, LGR6, and any combination thereof. In an embodiment, the living core potent cellular entities express RNA transcripts and/or polypeptides of one or more of Pax 7, Pax 3, MyoD, Myf 5, keratin 15, keratin 5, cluster of differentiation 34 (CD34), Sox9, c-Kit+, Sca-1+ or any combination thereof. In an embodiment, the supportive entities comprise mesenchymal derived cellular populations. In an embodiment, the supportive entities comprise cellular populations, extracellular matrix elements, or any combination thereof. In an embodiment, the extracellular matrix elements comprise one or more of hyaluronic acid, elastin, collagen, fibronectin, laminin, extracellular vesicles, enzymes, and glycoproteins.

In an embodiment, the stimulated biological material is derived from an osseous tissue interface. In an embodiment, the osseous tissue interface is a peri-cortical tissue interface, a peri-lamellar tissue interface, a peri-trabecular tissue interface, a cortico-cancellous tissue interface, or any combination thereof. In an embodiment, the stimulated biological material is derived from a triploblastic tissue interface.

In an embodiment, the composition further comprises an agent selected from the group consisting of a pharmaceutical, an enzyme, a molecule, and any combination thereof.

Also disclosed herein is a method for preparing the composition comprising stimulated biological material derived from an interface compartment, wherein the composition is capable of augmenting the generation or healing of a native tissue when administered to a subject in need thereof. The method comprises stimulating at least a portion of a mammalian interface compartment of a tissue specimen to generate stimulated biological material, wherein the mammalian interface compartment comprises a heterogeneous population of mammalian tissue interface cells. The method further comprises isolating a fraction of the stimulated biological material. In an embodiment, the fraction of the stimulated biological material is an acellular fraction.

In an embodiment, the portion of the mammalian interface compartment is stimulated using mechanical stimulation, chemical stimulation, enzymatic stimulation, energetic stimulation, electrical stimulation, biological stimulation, or any combination thereof. In an embodiment, the stimulating occurs in the presence of a biocompatible material. In an embodiment, the biocompatible material is selected from the group consisting of a pharmaceutical agent, an enzyme, a molecule, and combinations thereof. In an embodiment, the tissue specimen and the biocompatible material are in a volumetric ratio from about 1:1 to about 3:1.

In an embodiment, the method further comprises adding a biocompatible transfer agent to the stimulated biological material. In an embodiment, the biocompatible transfer agent is selected from alginate, gelatin, petroleum, collagen, mineral oil, hyaluronic acid, crystalloid, chondroitin sulfate, elastin, sodium alginate, silicone, PCL/ethanol, lecithin, a poloxamer, and any combination thereof.

In an embodiment, the tissue specimen is obtained from a plurality of donors.

In an embodiment, the method further comprises preserving the isolated fraction of the stimulated biological material. In an embodiment, the isolated fraction of the stimulated biological material is preserved via desiccation or cryodesiccation.

In an embodiment, the method further comprises adding a stabilizing agent to the isolated fraction of the stimulated biological material.

In an embodiment, the method further comprises incubating the stimulated portion of the mammalian interface compartment for about 12 to 72 hours prior to isolating the stimulated biological material. In an embodiment, the fraction of the stimulated biological material is isolated by centrifugation, filtration, or a combination thereof.

In an embodiment, the stimulating results in one or more alterations in interactomes of the heterogeneous population of mammalian tissue interface cells. In an embodiment, the isolated fraction of the stimulated biological material comprises a plurality of interactomes selected from among intracellular interactomes, intercellular interactomes, extracellular interactomes, transcellular interactomes, pericellular interactomes, and combinations thereof.

Disclosed herein is a process comprising disrupting an interface compartment of a tissue specimen to activate at least a portion of at least one interactome; and isolating an acellular composition from the disrupted interface compartment. In an embodiment, the tissue specimen is from triploblastic animal.

Also disclosed herein is a process comprising disrupting an interface compartment of a tissue specimen to activate and combine at least a portion of each of a plurality of interactomes; and isolating an acellular composition from the disrupted interface compartment. The plurality of interactomes can be selected from intracellular, intercellular, extracellular, transcellular, and pericellular interactomes, and combinations thereof.

In an embodiment, the tissue specimen is mammalian (e.g., rat, mouse, rabbit, pig, horse, human, goat, sheep, dog, cat, primate, cow, ox, camel, ass, guinea pig, or bison). The tissue specimen can be a plurality of tissue specimens from a plurality of donors. Alternatively, the tissue specimen can be one or more tissue specimens from a single donor.

The compositions disclosed herein can be preserved. For example, preserving can be accomplished by desiccating or cryodesiccating the composition.

A surfactant can be added to the compositions disclosed herein. A stabilizing agent can be added to the compositions disclosed herein. For example, the stabilizing agent can be selected from the group consisting of collagen, chondroitin sulphate, hydroxyapatite, crystalloids, organic solutions, molecules, elements, and combinations thereof.

The present disclosure is based upon the external and internal material interfaces which exist within and between grouped cellular entities. These interfaces are unique and dynamically interdependent to the collective totality of the complete interactome of each cell in a population and/or subpopulation. Each cell in this setting interfaces with a complex sub-network of materials surrounding it (e.g., including, but not limited to, other cells, extracellular matrices, substrates, agents, factors, and metabolites) which are further acted upon by non-static external gradients, forces, and systems.

Conventional approaches to biomaterials and/or biomedical engineering disregard these interactive complex sub-networks within and between cells (i.e., interactome(s)). The importance of the conventionally overlooked interactive complex sub-networks and/or the interactome(s) that exists in and/or between cellular entities within a system in maintaining, regulating, modulating and/or accelerating cell-tissue processes, pathways, and niche environments underlies the composition disclosed herein. The composition disclosed herein allows for such interactomes to combine and activate.

As aforementioned, the composition disclosed herein can also be referred to as a Composite-Interfacing Biomaterial Accelerant Substrate (CIBAS).

The CIBAS acts on responsive triploblastic-derived material systems by providing reactive agents to incomplete systems so as to complex and/or interact with agents of the incomplete system and/or partial sub-networks of the incomplete systems and thus accelerates functional product formation. Appropriate propagation of competent and/or functionally complete interface(s) and interactome(s) throughout intracellular, intercellular, extracellular, transcellular, and/or pericellular compartments is what results in generative, regenerative and/or neo-generative healing and/or restoration of functional self-propagating structure(s), which are capable of integration and/or association with greater system(s) in which such structure(s) were placed.

Functional product formation can be described as forming more organized structures, forming products within a reaction, and/or changing chemical, electrical, electrochemical and/or physical state(s) or status(es) of a material.

The CIBAS can alter the environment in which it is deployed by changing the environment through synthesis, alteration, modification, modulation, regulation, assembly or destruction of materials such as but not limited to genomic, epigenomic, transcriptomic, epitrascriptomic, proteomic, and/or epiproteomic materials, sub-cellular organelles or sub-cellular structures as well as derivatives of such structures, intracellular, intercellular extracellular, transcellular, and/or pericellular matrices, scaffolds, particles, fibers and or structural elements, anabolic, catabolic and/or metabolic processes and materials as well as derivatives of such materials, chemical, electrochemical and/or electrical environments, material mechanics, material forces, material kinetics and/or material thermodynamics, organic materials and/or living materials, tissue and/or organ systems, cell(s), cellular entities and/or cellular systems, and composite systems.

The CIBAS has a multitude of uses and applications spanning several fields of use, including but not limited, to medical, health, therapeutic, research, nonmedical, manufacturing, technology-related, defense-related, and nutritional uses. For example, the CIBAS can be used in clinical product applications in medicine such as applications related to the development of cell and/or tissue products, medical device(s), biologics products, therapeutics, small molecule products, and/or drug products. As another example, through integration, composition, and/or multi-material synthesis, the CIBAS can be combined with other technology or technologies for a combined product type. As a further example, the CIBAS can be used in applications related to the generation, regeneration, neogeneration, augmentation, alteration, assembly and/or destruction of cell, tissue and organ systems and/or derivatives thereof. As an example, the compositions disclosed herein can prevent or reduce scarring upon administration.

As another example, the CIBAS can be utilized in research applications and research related products (e.g., including but not limited to applications related to the development of research of clinical product types and combined technology and/or product types, applications related to the use of the invention for research products, research testing, research and development, applications related to the development of external life support, bioreactors, culture or maintenance of living materials).

As another example, the CIBAS can be utilized in applications for medical and/or non-medical efforts (e.g., including but not limited to pharmacological and/or cosmetic applications). For example, certain embodiments may modulate cell migration and proliferation, thereby reducing inflammation, accelerating wound healing, reducing scarring and ultimately promoting repair, regeneration and restoration of structure and function in all tissues. Certain embodiments may be provided directly, as a pre-treatment, as a pre-conditioning, coincident with injury, pre-injury or post-injury. Certain embodiments may reduce keloid scar formation pre- or post-cosmetic and/or clinical surgery. Certain embodiments may be used to treat internal injury caused by, but not limited to, disease or surgery to organs and tissues including but not limited to heart, bone, brain, spinal cord, retina, peripheral nerves and other tissues and organs commonly subject to acute and chronic injury or disease.

As a further example, the CIBAS can be used in the development of related technology derivatives, development of a transfer agent for other technologies, development of an activation or modulating agent for other technologies, and/or development of manufacturing or synthesis of small molecules, proteins, organelles or sub-cellular materials for organic or inorganic production.

As another example, the CIBAS can be used in the development of non-living materials.

As another example, the CIBAS can be used in applications related to the development of military, weapon, and/or defense derivatives.

As still another example, the CIBAS can be used in the development of food, nutrients, nourishments, nutraceuticals, and/or dietary supplements, and/or development of artificially intelligent, competent and/or propagating system(s) and/or unit(s) of a composite system(s).

Obtaining the composition involves disrupting an interface compartment to provide a peri-interfacing reactive material (PiRM), which is capable of assembling functional material (e.g., tissue). An embodiment of the composition is a targeted fraction of a reactive cellular progeny present at a peri-interface that is conducted away from the interface for processing.

The composition of the peri-interfacing reactive material (PiRM) includes materials of the interactome within and/or between the intracellular, intercellular, extracellular, transcellular, and/or pericellular compartments. The composition, in certain embodiments, includes components that do not naturally arrange into a single composition: cell-to-intracellular materials; cell-to-cell materials (i.e., intercellular); cell-to-extracellular materials; cell-to-transcellular materials; and cell-to-pericellular materials. The composition can be derived from an interface compartment within a tissue (e.g., cutaneous tissue) specimen.

An interface compartment can be obtained from a cell-tissue environment and/or multi-cellular environment and/or engineered cellular system(s) in either a complete interface compartment or sub-compartment interface.

A complete interface compartment refers to the content materials located within said region which when engineered as disclosed herein would supply or could supply, through further processing, those materials necessary for the development of the composition disclosed herein.

As described in more detail below, for each material substrate and/or tissue of interest, a complete interface compartment would include those essential components of that substrate and/or tissue that contribute to its unique functions or a component of such functions.

A sub-compartment interface also refers to the content materials located within said region which when engineered as disclosed herein would supply or could supply, through further processing, those materials necessary for the development of the composition disclosed herein. A sub-compartment interface refers to a portion of a complete interface compartment.

An interface compartment surrounding the triploblastic-derived material interface can be located with equipment available to those of ordinary skill in the art (e.g., via a laser scanning multi-photon confocal microscope). An interface compartment can be obtained through a variety of methods which would be understood by one of ordinary skill in the art, including but not limited to, common harvest, biopsy, punch, aspiration, cleavage, restriction, digestion, extraction, excision, disassociation, separation, removal, partition, and/or isolation protocols. Separation of the interface is complete when sufficient material is obtained for the application at hand, for example, volume/mass of material needed to treat the size of the wound.

The interface compartment is disrupted so as to dislocate such compartment and/or sub-compartment from the surrounding materials and alter the inherent organization of the material without complete destruction of the material and to obtain minimal polarization of the intracellular, intercellular, extracellular, transcellular and/or pericellular materials. As used herein, “minimal polarization” refers to the degree of polarization achieved by artificial manipulation of biological material that is necessary for a unit of tissue to be capable of assembling functional polarized tissue. Artificial manipulation may be achieved using mechanical, chemical, enzymatic, energetic, electrical, biological and/or other physical methods.

A variety of methods for disruption of target materials would be understood to those of skill in the art, including but not limited to, mechanical, chemical, enzymatic, energetic, electrical, biological and/or physical mechanisms. For example, targeted laser capture microscopy of material from the surrounding substances can produce the complete interface compartment or the sub-compartment interface. In an embodiment, the disrupting is accomplished by at least one of mechanically, physically, energetically, chemically, and electrically altering an inherent organization of the interface compartment.

In embodiments, disruption occurs in the presence of a biocompatible material. The biocompatible material may form various states of matter e.g., including but not limited to solids, liquids, and/or gases. In an embodiment, the biocompatible material is a solution (e.g., 0.9% NaCl, HBSS, PBS, DMEM, RPMI, lactated ringers, 5% dextrose in water, 3.2% sodium citrate). The biocompatible material can include an antibiotic such as an anti-Staphylococcal antibiotic (e.g., to alter microorganism population). In an embodiment, the biocompatible material is selected from the group consisting of a pharmaceutical agent, enzyme, molecule, and combinations thereof. The tissue specimen and the biocompatible material can be, for example, in a volumetric ratio from about 1:1 to about 1:2. Alternatively, the tissue specimen and the biocompatible material can be, for example, in a volumetric ratio from about 1:1 to about 2:1 or from about 1:1 to about 3:1. For example, the volumetric ratio can be about 1:1, about 2:1, or about 3:1.

Disrupting the interface compartment provides the peri-interfacing reactive material (PiRM) that is capable of assembling functional material (e.g., functional polarized tissue). In embodiments, the PiRM produced by the method described herein is capable of assembling functional material (e.g., functional polarized tissue) in vivo.

In embodiments, the PiRM produced by the method described herein is capable of assembling functional material (e.g., functional polarized tissue) ex vivo.

In embodiments, the PiRM produced by the method described herein is capable of assembling functional material (e.g., functional polarized tissue) in vitro.

During disruption of the interface compartment, acellular components of the intracellular, intercellular, extracellular, transcellular, and/or pericellular interactome(s) can be utilized to provide the composition.

After disruption of the interface compartment, the disrupted interface compartment can be incubated. Incubating can involve agitating the disrupted interface compartment, for example, for about 8 to about 12 hours. In certain embodiments, agitating the disrupted interface compartment can occur for about 8 to about 72 hours, for about 12 to about 72 hours, for about 24 to about 72 hours, for about 36 to about 72 hours, for about 48 to about 72 hours, or for about 60 to about 72 hours. Exemplary times include, but are not limited to, about 12 hours, about 24 hours, about 36 hours, about 48 hours, about 60 hours, and about 72 hours.

The composition can be isolated in a variety of ways known to those of ordinary skill in the art including, but not limited to, functional extravasation, filtration, fractionation, selective capture, selection, centrifugation, enrichment, ancillary reduction, separation, gradation, partition, pressurization, lysis, digestion, emulsification, protonation, and/or precipitation. As an example, isolating the composition can involve mechanical separation of the composition such as through centrifugation. As another example, isolation can also involve filtration of the composition such as after centrifugation. For example, filtration can involve passing the composition through an about 10 μm to about 100 μm filter. Filtration can involve passing the composition through an about 1 μm filter, an about 5 μm filter, an about 10 μm filter, an about 15 μm filter, an about 20 μm filter, an about 30 μm filter, an about 40 μm filter, an about 50 μm filter, an about 60 μm filter, an about 70 μm filter, and about 85 μm filter, an about 100 μm filter, an about 200 μm filter, an about 300 μm filter, an about 400 μm filter, or an about 500 μm filter.

As used herein, the term “accelerant” shall be understood to mean a substance used to accelerate a process.

As used herein, the term “acellular” shall be understood to mean essentially free of complete cells but may include a biologically insignificant level of complete cells and/or remaining cellular remnants such that the cells and/or remnants do not interfere with the properties of the composition. The degree of complete cell removal will depend on the exact source and methodology used to prepare the composition as well as the ultimate utility and desired state of the composition.

As used herein, the “administration” of a composition to a subject includes any route of introducing or delivering to a subject a composition to perform its intended function. Administration can be carried out by any suitable route, including but not limited to, by transplantation, orally, intranasally, parenterally (intravenously, intramuscularly, intraperitoneally, or subcutaneously), rectally, intrathecally, or topically. Administration includes self-administration and the administration by another. Exemplary methods of administration include, but are not limited to, injection, topical application, coating, and impregnation.

The term “biomaterial” shall be understood to mean any substance or combination of substances, other than drugs, synthetic or natural in origin, which can be used for any period of time, which augments or replaces partially or totally any tissue, organ or function of the body, in order to maintain or improve the quality of life of an individual.

Unless indicated otherwise, as used herein, the term “composite” shall be understood to mean comprised of a plurality of parts or elements.

As used herein, “core potent cellular entities” refer to cellular entities that are capable of intercellular communication, migration, chemotaxis, proliferation, differentiation, transdifferentiation, dedifferentiation, transient amplification, asymmetrical division and include stem cells, progenitor cells, and transit-amplifying cells. Core potent cellular entities may be identified or established by, for example, assaying for certain sub-cellular biomarkers (i.e., DNA, RNA, and proteins). In some embodiments, core potent cellular entities express RNA transcripts and/or polypeptides of one or more Leucine Rich Repeat Containing G Protein-Coupled Receptors (LGR), such as LGR4, LGR5, LGR6, or combinations thereof. Additionally or alternatively, in some embodiments, core potent cellular entities express RNA transcripts and/or polypeptides of one or more of Pax 7, Pax 3, MyoD, Myf 5, keratin 15, keratin 5, cluster of differentiation 34 (CD34), Sox9, c-Kit+, Sca-1+, and any combination thereof. Additional examples of biomarkers for core potent cellular entities are described in Wong et al., International Journal of Biomaterials, vol. 2012, Article ID 926059, 8 pages, 2012.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease or condition and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein.

As used herein, “extracellular matrix” and “extracellular matrix elements” refer to extracellular macromolecules, such as hyaluronic acid, elastin, collagen, fibronectin, laminin, extracellular vesicles, enzymes, and glycoproteins, that are organized as a three-dimensional network to provide structural and biochemical support for surrounding cells.

As used herein, the terms “functional material”, “functional tissue”, and “functional polarized tissue” refer to an ensemble of cells and their extracellular matrix having the same origin and executing biological functions similar to that observed in the native counterpart tissue. In some embodiments, the “functional material”, “functional tissue”, or “functional polarized tissue” exhibits characteristics such polarity, density, flexibility, etc., similar to that observed in the native counterpart tissue.

As used herein, the term “interactome” refers to a set of molecular interactions which occur within and/or between a cell or cellular material. Examples of interactomes include, but are not limited to, the intracellular, intercellular, extracellular, transcellular, and pericellular interactomes.

The term “interface” shall be understood to mean the region of contact between living and/or organic material and other biomaterial or organic/inorganic material.

As used herein, the term “interface compartment” refers to a portion of a tissue specimen that contains a tissue interface.

As used herein, the term “material interface” refers to the region, area and/or location where two or more different or distinguishable cells approach, contact, merge, integrate, incorporate, unite, coalesce, combine, compound, fuse, abut, touch, border, meld, communicate, synapse, junction, interact, share, aggregate, connect, penetrate, surround, or form with each other in an environment and/or system which may or may not contain other materials, substrates or factors. This other environment(s) and/or system(s) may be used to interact with the compositions disclosed herein.

As used herein, “stimulated” refers to activating (e.g., changing) the physiological state of heterogeneous mammalian tissue/cells present at a tissue interface that can be performed by one or a combination of signals including electrical stimulation, oxygen gradient, chemokine receptor binding, paracrine receptor binding, cell membrane alteration, cytoskeletal alteration, physical manipulation of cells, alteration of physiological gradients, alteration of temperature, small molecule interactions, introduction of nucleotides and ribonucleotides such as small inhibitory RNAs, which are sufficient to induce one or more of the following phenotypes/outcomes: altered gene expression, altered protein translation, altered intracellular and intercellular signaling, altered binding of vesicles to membranes, altered ATP production and consumption, and altered cellular mobility.

The term “substrate” shall be understood to mean the surface or material on or from which an organism lives, grows, or obtains its nourishment.

As used herein, “supportive entities” refer to non-stem cell populations (e.g., supportive cellular entities) and/or extracellular matrix materials that provide structural and biochemical support for core potent cellular entities. In some embodiments, supportive cellular entities may comprise proliferating and/or differentiating cells. Additionally or alternatively, in some embodiments, supportive cellular entities may be identified by expression of biomarkers such as BMPr1a, BMP2, BMP6, FGF, Notch receptors, Delta ligands, CXCL12, Sonic Hedge Hog, VEGF, TGFβ, Wnt, HGF, NG2, and alpha smooth muscle actin. In some embodiments, the supportive cellular entities comprise mesenchymal derived cellular populations.

As used herein, a “tissue interface” refers to a location at which independent and optionally unrelated tissue systems interact and communicate with each other. In some embodiments, components of a tissue interface currently promote/promoted histogenesis and cell development and/or metabolism, including but not limited to proliferation, differentiation, migration, anabolism, catabolism, stimulation, or at least one of intracellular, intercellular, extracellular, transcellular, and pericellular communication or any combination thereof.

Exemplary tissue interfaces include, but are not limited to, blastomeric apical cellular interfaces, blastomeric lateral cellular interfaces, blastomeric basal cellular interfaces, ectodermal apical cellular interfaces, ectodermal lateral cellular interfaces, ectodermal basal cellular interfaces, mesodermal apical cellular interfaces, mesodermal lateral cellular interfaces, mesodermal basal cellular interfaces, endodermal apical cellular interfaces, endodermal lateral cellular interfaces, endodermal basal cellular interfaces, cutaneous tissue interface, an osseous tissue interface, a musculoskeletal tissue interface, a smooth muscle tissue interface, a cardiac muscle tissue interface, a cartilage tissue interface, an adipose tissue interface, a gastrointestinal tissue interface, a pulmonary tissue interface, a esophageal tissue interface, a gastric tissue interface, a renal tissue interface, a hepatic tissue interface, a pancreatic tissue interface, a blood vessel tissue interface, a lymphatic tissue interface, a central nervous tissue interface, a urogenital tissue interface, a glandular tissue interface, a dental tissue interface, a peripheral nerve tissue interface, a birth tissue interface, and an optic tissue interface.

A cutaneous tissue interface can include an epithelial-dermal tissue interface, a papillary dermal-reticular dermal tissue interface, a dermal-hypodermal interface, a hypodermal-subdermal interface, or any combination thereof.

An osseous tissue interface can include a peri-cortical tissue interface, a peri-lamellar tissue interface, a peri-trabecular tissue interface, a cortico-cancellous tissue interface, or any combination thereof.

A musculoskeletal tissue interfaces can include a myo-epimysial tissue interface, a myo-perimysial tissue interface, a myo-endomysial tissue interface, a myo-fascial tissue interface, a tendon-muscle tissue interface, a tendon-bone tissue interface, a ligament-bone tissue interface, or any combination thereof.

A smooth muscle tissue interface can include a perivascular tissue interface, a perivisceral tissue interface, a perineural tissue interface, or any combination thereof.

A cardiac muscle tissue interface can include an endocardial-myocardial tissue interface, a myocardial-epicardial tissue interface, an epicardial-pericardial tissue interface, a pericardial-adipose tissue interface, or any combination thereof.

A cartilage tissue interface can include a chondrial-perichondrial tissue interface, a chondrial-endochondrial tissue interface, an endochondrial-subchondral bone interface, a chondrial-endochondrial bone interface, an endochondrial-subchondral bone interface, or any combination thereof.

An adipose tissue interface can include an adipo-perivascular tissue interface, an adipo-peristromal tissue interface, or any combination thereof.

A gastrointestinal tissue (small and large intestinal) interface can include a mucosal-submucosal tissue interface, a sub-mucosal-muscularis tissue interface, a muscularis-serosal tissue interface, a serosal-mesentery tissue interface, a myo-neural tissue interface, a submucosal-neural tissue interface, or any combination thereof.

A pulmonary tissue interface can include a mucosal-submucosal tissue interface, a sub-mucosal-muscularis tissue interface, a sub-mucosal-cartilage tissue interface, a muscular-adventitial tissue interface, a ductal-adventitial tissue interface, a parenchymal-serosal tissue interface, a serosal-mesentery tissue interface, a myo-neural tissue interface, a submucosal-neural tissue interface, or any combination thereof.

An esophageal tissue interface can include a mucosal-submucosal tissue interface, a sub-mucosal-muscularis tissue interface, a muscularis-adventitial tissue interface, a myo-neural tissue interface, a submucosal-neural tissue interface, or any combination thereof.

A gastric tissue interfaces can include a mucosal-submucosal tissue interface, a sub-mucosal-muscularis tissue interface, a muscularis-serosal tissue interface, a myo-neural tissue interface, a submucosal-neural tissue interface, or any combination thereof.

A renal tissue interface can include a capsule-cortical tissue interface, a cortical-medullary tissue interface, a neuro-parenchymal tissue interface, or any combination thereof.

A hepatic tissue interface can include a ductal epithelial-parenchymal tissue interface.

A pancreatic tissue interface can include a ductal epithelial-parenchymal tissue interface, a glandular epithelial-parenchymal tissue interface, or any combination thereof.

A blood vessel tissue interface can include an endothelial-tunica tissue interface, a tunica-tunica tissue interface, or any combination thereof.

A lymphatic tissue (lymph node, spleen, thymus) interface can include a cortico-medullary tissue interface, a medullary-capsule tissue interface, a capsule-pulp tissue interface, or any combination thereof.

A central nervous tissue interface can include a dural-cortex tissue interface, a cortical grey matter-medullary white matter tissue interface, a meningeal-neural tissue interface, or any combination thereof.

A urogenital tissue interface can include an epithelial-mucosal tissue interface, a mucosal-muscular tissue interface, a muscular-adventitial tissue interface, a corporal-vascular tissue interface, a corporal-muscular tissue interface, or any combination thereof.

A glandular tissue interface can include an epithelial-parenchymal tissue interface.

A dental tissue interface can include a dentin-pulp tissue interface.

A peripheral nerve tissue interface can include an epineural-perineural tissue interface, a perineural-endoneural tissue interface, an endoneural-axonal tissue interface, or any combination thereof.

A birth tissue interface can include an amnion-fluid tissue interface, an epithelial-sub-epithelial tissue interface, an epithelial-stroma tissue interface, a compact-fibroblast tissue interface, a fibroblast-intermediate tissue interface, an intermediate-reticular tissue interface, an amnio-chroion tissue interface, a reticular-trophoblast tissue interface, a trophoblast-uterine tissue interface, a trophoblast-decidua tissue interface, or any combination thereof.

An optic tissue interface can include an epithelial-membrane tissue interface, a membrane-stroma tissue interface, a stromal-membrane tissue interface, a membrane-endothelial tissue interface, an endothelial-fluid tissue interface, a scleral-choroid tissue interface, a choroid-epithelial tissue interface, an epithelial-segmental photoreceptor tissue interface, a segmental photoreceptor-membrane tissue interface, a membrane-outer nuclear layer tissue interface, an outer nuclear layer-outer plexiform tissue interface, an outer plexiform-inner plexiform tissue interface, an inner plexiform-ganglion tissue interface, a ganglion-neural fiber tissue interface, a neural fiber tissue interface-membrane tissue interface, or any combination thereof.

In embodiments, the CIBAS is the isolated composition. In other embodiments, the isolated composition is modified to provide the CIBAS.

A biocompatible transfer agent can be added to the composition. For example, the composition can be formulated with a biocompatible transfer agent into, e.g., including but not limited to an injectable formulation, a topical liquid formulation, a topical gel formulation, a serum, an ointment, a foam, a cream, a paste, a lotion, or a powder. Exemplary biocompatible transfer agents include an alginate, gelatin, petroleum, collagen, mineral oil, hyaluronic acid, crystalloid, chondroitin sulfate, elastin, sodium alginate, silicone, PCL/ethanol, lecithin, a poloxamer, 1×HBSS, 10×HBSS, 1×PBS/DPBS, 10×PBS/DPBS, 10×DMEM, RPMI, saline, saline sodium citrate, sodium citrate, citric acid, and any combination thereof. The composition may be combined with a pharmaceutically acceptable surfactant (e.g., a wetting agent, an emulsifying agent, a suspending agent, etc.).

The biocompatible transfer agent can contain one or more components in which organic materials may subsist and/or exist. As such, biocompatible transfer agents may include but are not limited to solids, liquids, gases in which organic materials may be placed and subsist and/or exist.

In embodiments, the composition may comprise material derived from a single tissue type, for example, adipose, bone, brain, spinal cord, cartilage, heart, liver, muscle, pancreas, skin, or tendon.

In certain embodiments, the composition may comprise material derived from a plurality of different tissue types, for example bone and muscle, and blood clot/serum and bone, etc.

In certain embodiments, the composition can undergo further treatment(s) (e.g., freeze-drying, dialysis, rinsing, heat curing, cross-linking (e.g., with EDC/NHS, glutaraldehyde, or calcium chloride), desiccating, molding/texturizing, electrospinning, or any combination thereof). As another example, the composition can be desiccated or cryodesiccated (i.e., freeze-dried). Desiccation and cryodesiccation are exemplary preservation methods. As another example, the composition can include an additional therapeutic agent (e.g., small molecule). As yet another example, the composition (isolated or formulated) can be added to an absorbable wound dressing (e.g., mesh, gauze, cotton, foam, tape, collagen, sponge, matrix, or bandage). The composition may also contain a sequence recognized within the Leucine-rich repeat-containing G-protein coupled receptor family (LGR) or an agent which interfaces with this family of sequences.

As another example, the composition (isolated or formulated) can be added to a biocompatible substrate. For example, a 3D printed bone scaffold can be soaked in the isolated composition. Further, for example, an electrospun bone scaffold can be soaked in the composition. Electrospinning is a process whereby a fibrous structure is produced by means of forcing and elongating the draw of electrically charged thread(s) of polymer solutions or “melts”, commonly in diameters of a few hundred nanometers. Incorporation of bioactive components onto electrospun fibrous structure(s) can include physically soaking electrospun fibers in solution(s) comprising bioactive components.

The compositions disclosed herein can serve as a substitute for scaffold or void fillers or in conjunction with other devices to promote tissue healing, fill voids, maintain essential structure, and bridge separate tissue surfaces via its biologic and mechanical characteristics. Thus, the compositions disclosed herein can be applied in graft procedures including, but not limited to, orthopedic surgery, neurological surgery, plastic surgery, dental surgery, and dermatologic surgery.

The compositions disclosed herein can serve as a media to support cell proliferation in a cell or tissue culture in vitro or ex vivo. Stabilized compositions disclosed herein are useful as a scaffold or matrix for a cell or tissue culture in vitro or ex vivo. As media or stabilized compositions for cell or tissue culture, the compositions disclosed herein are useful in research and development in tissue engineering and regenerative medicine.

The compositions disclosed herein can be autologous. Alternatively, the compositions disclosed herein can be allogeneic. Alternatively, the compositions disclosed herein can be xenogeneic.

In embodiments, the compositions disclosed herein are characterized by nanoparticle histogram profiling. The histogram typically shows the distribution and size of a population of nanoparticles, including naturally occurring nanoparticles such as exosomes, as well as the concentration of nanoparticle size over a specific range. The histogram can comprise no mode, one mode, or multiple modes. Histogram “peaks” or “modes” typically represent the value(s) or data range(s) that appear with the most frequency (concentration) in a given profile.

In other embodiments, the compositions disclosed herein are characterized by Raman spectroscopy. The Raman spectrum is typically represented by a diagram plotting the Raman intensity versus the Raman shift of the peaks. The “peaks” of Raman spectroscopy are also known as “absorption bands”. The characteristic peaks of a given Raman spectrum can be selected according to the peak locations and their relative intensity.

One of ordinary skill in the art recognizes that the measurements of the Raman peak shifts and/or intensity for a given composition will vary within a margin of error. The values of peak shift, expressed in reciprocal wave numbers (cm⁻¹), allow appropriate error margins. Typically, the error margins are represented by “±”. For example, the Raman shift of about “1310±10” denotes a range from about 1310+10, i.e., about 1320, to about 1310−10, i.e., about 1300. Depending on the sample preparation techniques, the calibration techniques applied to the instruments, human operational variations, etc., one of ordinary skill in the art recognizes that the appropriate error of margins for a Raman shift can be ±12; ±10; ±8; ±5; ±4, ±3, ±1, or less.

Additional details of the methods and equipment used for the Raman spectroscopy analysis are described in the Examples section.

In embodiments, the composition exhibits a Raman spectrum comprising peaks at about 856±4 cm⁻¹, about 965±4 cm⁻¹, about 1446±4 cm⁻¹, about 1656±4 cm⁻¹, and about 2900±4 cm⁻¹. In embodiments, the composition exhibits a Raman spectrum comprising peaks at about 856±12 cm⁻¹, about 965±12 cm⁻¹, about 1446±12 cm⁻¹, about 1656±12 cm⁻¹, and about 2900±12 cm⁻¹. In embodiments, the composition exhibits a Raman spectrum comprising peaks at about 856±10 cm⁻¹, about 965±10 cm⁻¹, about 1446±10 cm⁻¹, about 1656±10 cm⁻¹, and about 2900±10 cm⁻¹. In embodiments, the composition exhibits a Raman spectrum comprising peaks at about 856±8 cm⁻¹, about 965±8 cm⁻¹, about 1446±8 cm⁻¹, about 1656±8 cm⁻¹, and about 2900±8 cm⁻¹. In embodiments, the composition exhibits a Raman spectrum comprising peaks at about 856±5 cm⁻¹, about 965±5 cm⁻¹, about 1446±5 cm⁻¹, about 1656±5 cm⁻¹, and about 2900±5 cm⁻¹. In embodiments, the composition exhibits a Raman spectrum comprising peaks at about 856±3 cm⁻¹, about 965±3 cm⁻¹, about 1446±3 cm⁻¹, about 1656±3 cm⁻¹, and about 2900±3 cm⁻¹. In embodiments, the composition exhibits a Raman spectrum comprising peaks at about 856±1 cm⁻¹, about 965±1 cm⁻¹, about 1446±1 cm⁻¹, about 1656±1 cm⁻¹, and about 2900±1 cm⁻¹.

In embodiments, the composition has a Raman spectrum comprising peaks listed in Table 1A, 1B, 1C, 1D, 1E, 1F, or 1G.

| | | | | Table | Table | Table |
|---|---|---|---|---|---|---|
| Table 1A | Table 1B | Table 1C | Table 1D | 1E | 1F | 1G |
| 856 ± 4 cm⁻¹ | 856 ± 12 cm⁻¹ | 856 ± 10 cm⁻¹ | 856 ± 8 cm⁻¹ | 856 ± 5 cm⁻¹ | 856 ± 3 cm⁻¹ | 856 ± 1 cm⁻¹ |
| 965 ± 4 cm⁻¹ | 965 ± 12 cm⁻¹ | 965 ± 10 cm⁻¹ | 965 ± 8 cm⁻¹ | 965 ± 5 cm⁻¹ | 965 ± 3 cm⁻¹ | 965 ± 1 cm⁻¹ |
| 1248 ± 4 cm⁻¹ | 1248 ± 12 cm⁻¹ | 1248 ± 10 cm⁻¹ | 1248 ± 8 cm⁻¹ | 1248 ± 5 cm⁻¹ | 1248 ± 3 cm⁻¹ | 1248 ± 1 cm⁻¹ |
| 1300 ± 4 cm⁻¹ | 1300 ± 12 cm⁻¹ | 1300 ± 10 cm⁻¹ | 1300 ± 8 cm⁻¹ | 1300 ± 5 cm⁻¹ | 1300 ± 3 cm⁻¹ | 1300 ± 1 cm⁻¹ |
| 1345 ± 4 cm⁻¹ | 1345 ± 12 cm⁻¹ | 1345 ± 10 cm⁻¹ | 1345 ± 8 cm⁻¹ | 1345 ± 5 cm⁻¹ | 1345 ± 3 cm⁻¹ | 1345 ± 1 cm⁻¹ |
| 1448 ± 4 cm⁻¹ | 1448 ± 12 cm⁻¹ | 1448 ± 10 cm⁻¹ | 1448 ± 8 cm⁻¹ | 1448 ± 5 cm⁻¹ | 1448 ± 3 cm⁻¹ | 1448 ± 1 cm⁻¹ |
| 1586 ± 4 cm⁻¹ | 1586 ± 12 cm⁻¹ | 1586 ± 10 cm⁻¹ | 1586 ± 8 cm⁻¹ | 1586 ± 5 cm⁻¹ | 1586 ± 3 cm⁻¹ | 1586 ± 1 cm⁻¹ |
| 1657 ± 4 cm⁻¹ | 1657 ± 12 cm⁻¹ | 1657 ± 10 cm⁻¹ | 1657 ± 8 cm⁻¹ | 1657 ± 5 cm⁻¹ | 1657 ± 3 cm⁻¹ | 1657 ± 1 cm⁻¹ |
| 2900 ± 4 cm⁻¹ | 2900 ± 12 cm⁻¹ | 2900 ± 10 cm⁻¹ | 2900 ± 8 cm⁻¹ | 2900 ± 5 cm⁻¹ | 2900 ± 3 cm⁻¹ | 2900 ± 1 cm⁻¹ |

In embodiments, the composition has a Raman spectrum comprising peaks listed in Table 2A, 2B, 2C, 2D, 2E, 2F, or 2G.

| Table 2A | Table 2B | Table 2C | Table 2D | Table 2E | Table 2F | Table 2G |
|---|---|---|---|---|---|---|
| 856 ± 4 cm⁻¹ | 856 ± 12 cm⁻¹ | 856 ± 10 cm⁻¹ | 856 ± 8 cm⁻¹ | 856 ± 5 cm⁻¹ | 856 ± 3 cm⁻¹ | 856 ± 1 cm⁻¹ |
| 965 ± 4 cm⁻¹ | 965 ± 12 cm⁻¹ | 965 ± 10 cm⁻¹ | 965 ± 8 cm⁻¹ | 965 ± 5 cm⁻¹ | 965 ± 3 cm⁻¹ | 965 ± 1 cm⁻¹ |
| 1076 ± 4 cm⁻¹ | 1076 ± 12 cm⁻¹ | 1076 ± 10 cm⁻¹ | 1076 ± 8 cm⁻¹ | 1076 ± 5 cm⁻¹ | 1076 ± 3 cm⁻¹ | 1076 ± 1 cm⁻¹ |
| 1300 ± 4 cm⁻¹ | 1300 ± 12 cm⁻¹ | 1300 ± 10 cm⁻¹ | 1300 ± 8 cm⁻¹ | 1300 ± 5 cm⁻¹ | 1300 ± 3 cm⁻¹ | 1300 ± 1 cm⁻¹ |
| 1446 ± 4 cm⁻¹ | 1446 ± 12 cm⁻¹ | 1446 ± 10 cm⁻¹ | 1446 ± 8 cm⁻¹ | 1446 ± 5 cm⁻¹ | 1446 ± 3 cm⁻¹ | 1446 ± 1 cm⁻¹ |
| 1655 ± 4 cm⁻¹ | 1655 ± 12 cm⁻¹ | 1655 ± 10 cm⁻¹ | 1655 ± 8 cm⁻¹ | 1655 ± 5 cm⁻¹ | 1655 ± 3 cm⁻¹ | 1655 ± 1 cm⁻¹ |
| 2900 ± 4 cm⁻¹ | 2900 ± 12 cm⁻¹ | 2900 ± 10 cm⁻¹ | 2900 ± 8 cm⁻¹ | 2900 ± 5 cm⁻¹ | 2900 ± 3 cm⁻¹ | 2900 ± 1 cm⁻¹ |

In embodiments, the composition has a Raman spectrum comprising peaks listed in Table 3A, 3B, 3C, 3D, 3E, 3F, or 3G.

| Table 3A | Table 3B | Table 3C | Table 3D | Table 3E | Table 3F | Table 3G |
|---|---|---|---|---|---|---|
| 856 ± 4 cm⁻¹ | 856 ± 12 cm⁻¹ | 856 ± 10 cm⁻¹ | 856 ± 8 cm⁻¹ | 856 ± 5 cm⁻¹ | 856 ± 3 cm⁻¹ | 856 ± 1 cm⁻¹ |
| 965 ± 4 cm⁻¹ | 965 ± 12 cm⁻¹ | 965 ± 10 cm⁻¹ | 965 ± 8 cm⁻¹ | 965 ± 5 cm⁻¹ | 965 ± 3 cm⁻¹ | 965 ± 1 cm⁻¹ |
| 1000 ± 4 cm⁻¹ | 1000 ± 12 cm⁻¹ | 1000 ± 10 cm⁻¹ | 1000 ± 8 cm⁻¹ | 1000 ± 5 cm⁻¹ | 1000 ± 3 cm⁻¹ | 1000 ± 1 cm⁻¹ |
| 1129 ± 4 cm⁻¹ | 1129 ± 12 cm⁻¹ | 1129 ± 10 cm⁻¹ | 1129 ± 8 cm⁻¹ | 1129 ± 5 cm⁻¹ | 1129 ± 3 cm⁻¹ | 1129 ± 1 cm⁻¹ |
| 1295 ± 4 cm⁻¹ | 1295 ± 12 cm⁻¹ | 1295 ± 10 cm⁻¹ | 1295 ± 8 cm⁻¹ | 1295 ± 5 cm⁻¹ | 1295 ± 3 cm⁻¹ | 1295 ± 1 cm⁻¹ |
| 1448 ± 4 cm⁻¹ | 1448 ± 12 cm⁻¹ | 1448 ± 10 cm⁻¹ | 1448 ± 8 cm⁻¹ | 1448 ± 5 cm⁻¹ | 1448 ± 3 cm⁻¹ | 1448 ± 1 cm⁻¹ |
| 1656 ± 4 cm⁻¹ | 1656 ± 12 cm⁻¹ | 1656 ± 10 cm⁻¹ | 1656 ± 8 cm⁻¹ | 1656 ± 5 cm⁻¹ | 1656 ± 3 cm⁻¹ | 1656 ± 1 cm⁻¹ |
| 2900 ± 4 cm⁻¹ | 2900 ± 12 cm⁻¹ | 2900 ± 10 cm⁻¹ | 2900 ± 8 cm⁻¹ | 2900 ± 5 cm⁻¹ | 2900 ± 3 cm⁻¹ | 2900 ± 1 cm⁻¹ |

In embodiments, the composition has a Raman spectrum comprising peaks listed in Table 4A, 4B, 4C, 4D, 4E, 4F, or 4G.

| Table 4A | Table 4B | Table 4C | Table 4D | Table 4E | Table 4F | Table 4G |
|---|---|---|---|---|---|---|
| 856 ± 4 cm⁻¹ | 856 ± 12 cm⁻¹ | 856 ± 10 cm⁻¹ | 856 ± 8 cm⁻¹ | 856 ± 5 cm⁻¹ | 856 ± 3 cm⁻¹ | 856 ± 1 cm⁻¹ |
| 965 ± 4 cm⁻¹ | 965 ± 12 cm⁻¹ | 965 ± 10 cm⁻¹ | 965 ± 8 cm⁻¹ | 965 ± 5 cm⁻¹ | 965 ± 3 cm⁻¹ | 965 ± 1 cm⁻¹ |
| 1000 ± 4 cm⁻¹ | 1000 ± 12 cm⁻¹ | 1000 ± 10 cm⁻¹ | 1000 ± 8 cm⁻¹ | 1000 ± 5 cm⁻¹ | 1000 ± 3 cm⁻¹ | 1000 ± 1 cm⁻¹ |
| 1445 ± 4 cm⁻¹ | 1445 ± 12 cm⁻¹ | 1445 ± 10 cm⁻¹ | 1445 ± 8 cm⁻¹ | 1445 ± 5 cm⁻¹ | 1445 ± 3 cm⁻¹ | 1445 ± 1 cm⁻¹ |
| 1656 ± 4 cm⁻¹ | 1656 ± 12 cm⁻¹ | 1656 ± 10 cm⁻¹ | 1656 ± 8 cm⁻¹ | 1656 ± 5 cm⁻¹ | 1656 ± 3 cm⁻¹ | 1656 ± 1 cm⁻¹ |
| 2900 ± 4 cm⁻¹ | 2900 ± 12 cm⁻¹ | 2900 ± 10 cm⁻¹ | 2900 ± 8 cm⁻¹ | 2900 ± 5 cm⁻¹ | 2900 ± 3 cm⁻¹ | 2900 ± 1 cm⁻¹ |

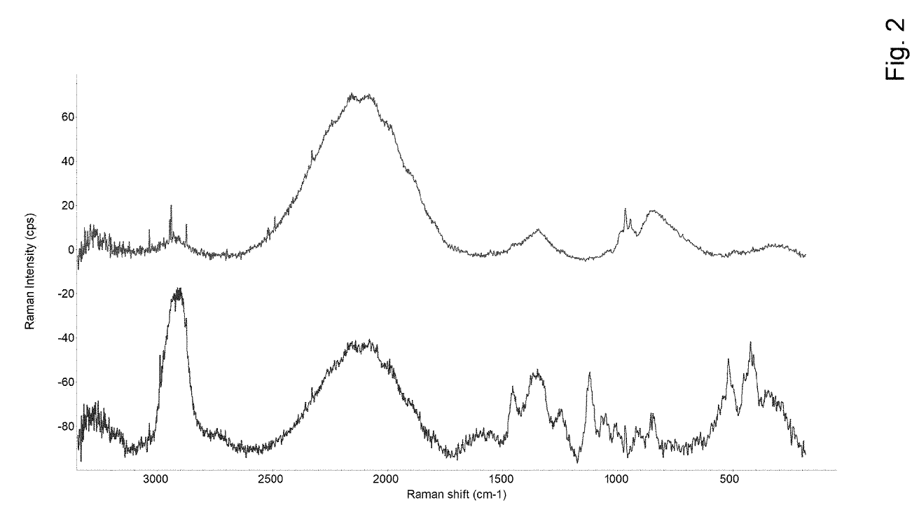

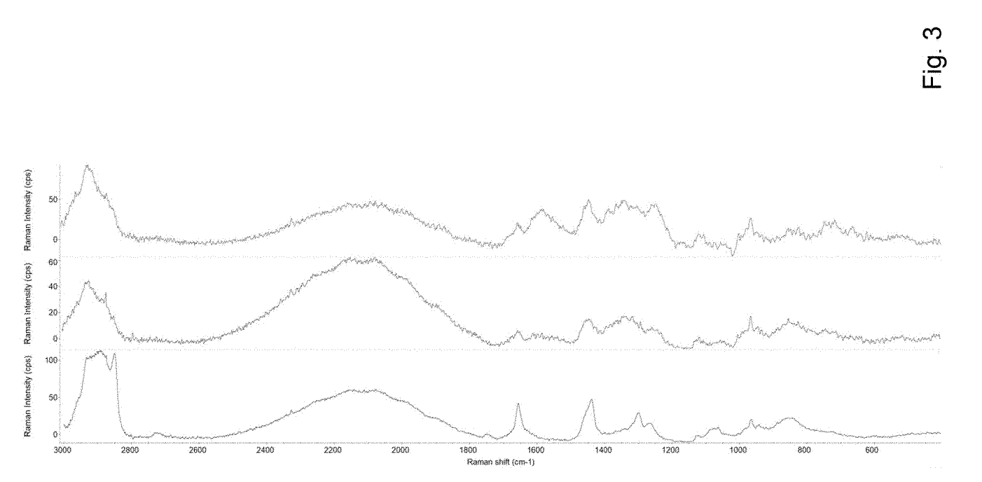

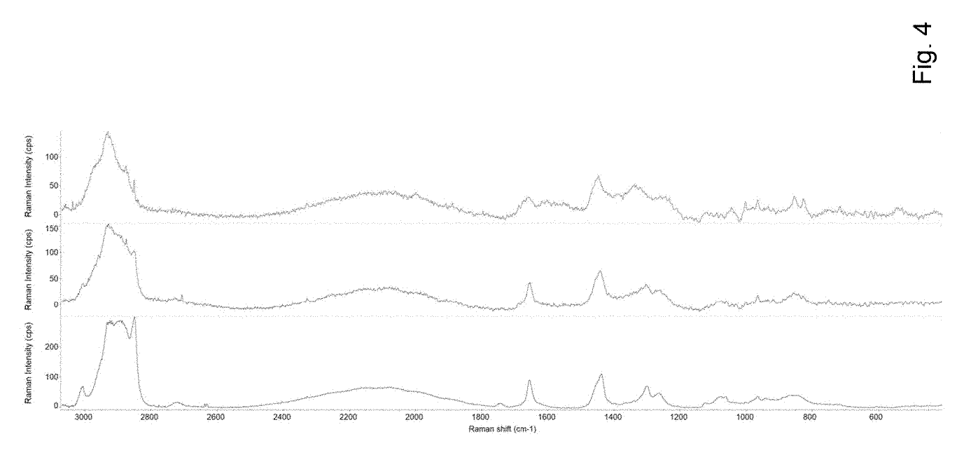

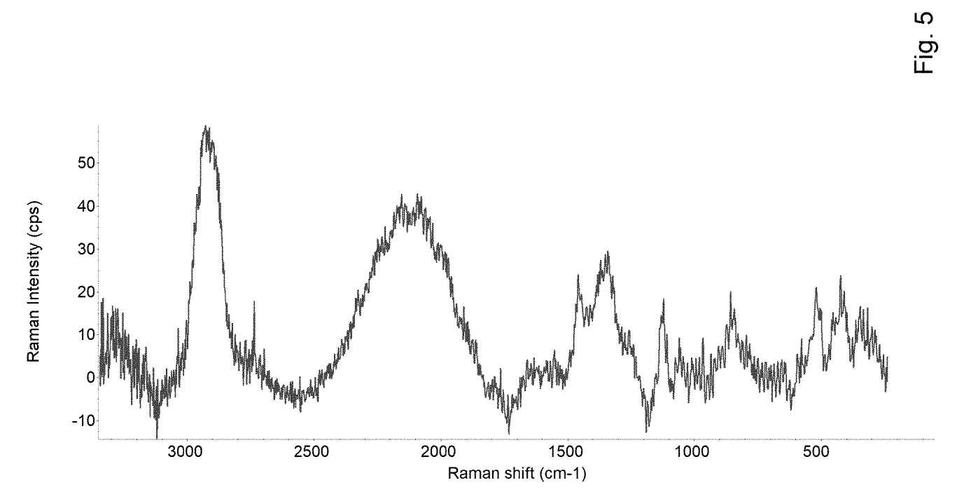

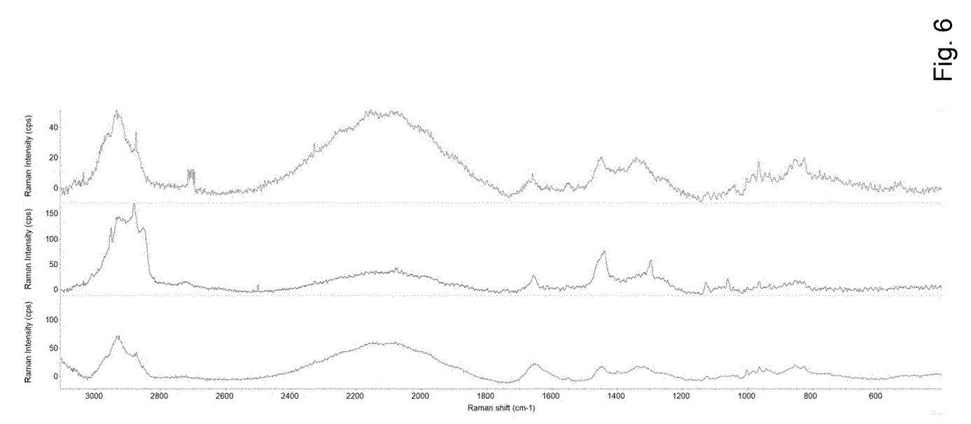

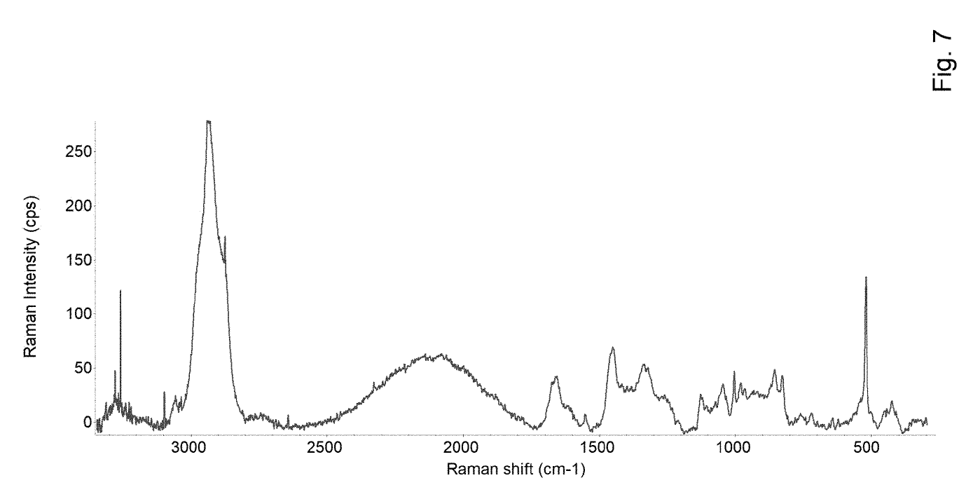

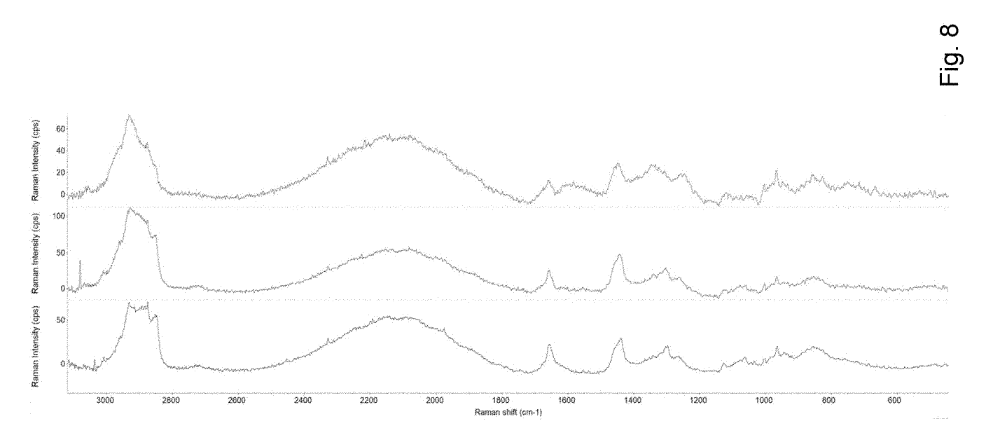

In embodiments, the composition exhibits a Raman spectrum that is substantially similar to one of the Raman spectra of FIG. 2, FIG. 3, FIG. 4, FIG. 5, FIG. 6, FIG. 7 and FIG. 8. In an embodiment, the composition exhibits a Raman spectrum that is substantially similar to one of the Raman spectra of FIG. 2. In an embodiment, the composition exhibits a Raman spectrum that is substantially similar to one of the Raman spectra of FIG. 3. In an embodiment, the composition exhibits a Raman spectrum that is substantially similar to one of the Raman spectra of FIG. 4. In an embodiment, the composition exhibits a Raman spectrum that is substantially similar to the Raman spectrum of FIG. 5. In an embodiment, the composition exhibits a Raman spectrum that is substantially similar to one of the Raman spectra of FIG. 6. In an embodiment, the composition exhibits a Raman spectrum that is substantially similar to the Raman spectrum of FIG. 7. In an embodiment, the composition exhibits a Raman spectrum that is substantially similar to one of the Raman spectra of FIG. 8.

Also disclosed herein is a kit comprising a composition as disclosed herein and instructions for use.

Further disclosed herein is a method for augmenting tissue regeneration in a subject in need thereof comprising administering to the subject an effective amount of a composition as disclosed herein.

Additionally disclosed herein is a method for augmenting healing of native tissue a subject in need thereof comprising administering to the subject an effective amount of a composition as disclosed herein. In an embodiment, the native tissue is skin and administration of the composition prevents or reduces scarring in the subject.

In an embodiment, the subject is suffering from a degenerative bone disease. In an embodiment, the degenerative bone disease is osteoarthritis or osteoporosis. In an embodiment, the subject is suffering from a bone fracture or break. In an embodiment, the fracture is a stable fracture, an open compound fracture, a transverse fracture, an oblique fracture, or a comminuted fracture.

### Exemplary Embodiments

1. A process, comprising the steps of:
disrupting an interface compartment of a tissue specimen to activate and combine at least a portion of each of a plurality of interactomes; and
isolating an acellular composition from the disrupted interface compartment.
2. The process of any preceding claim, wherein the disrupting occurs in the presence of a biocompatible material.
3. The process of any preceding claim, wherein the biocompatible material is selected from the group consisting of a pharmaceutical agent, enzyme, molecule, and combinations thereof.
4. The process of any preceding claim, further comprising the step of adding a biocompatible transfer agent to the composition.
5. The process of any preceding claim, further comprising the step of preserving the composition.
6. The process of any preceding claim, further comprising the step of incubating the disrupted interface compartment.
7. The process of any preceding claim, wherein the tissue specimen is mammalian.
8. The process of any preceding claim, wherein the tissue specimen comprises a plurality of tissue specimens from a plurality of donors.
9. The process of any preceding claim, wherein the tissue specimen and the biocompatible material are in a volumetric ratio from about 1:1 to about 1:2.
10. The process of any preceding claim, wherein the volumetric ratio is about 1:1.
11. The process of any preceding claim, wherein the disrupting is accomplished by at least one of mechanically, physically, energetically, chemically, and electrically altering an inherent organization of the interface compartment.
12. The process of any preceding claim, wherein the preserving is accomplished by desiccating or cryodesiccating the composition.
13. The process of any preceding claim, further comprising the step of adding a surfactant to the composition.
14. The process of any preceding claim, further comprising the step of adding a stabilizing agent to the composition.
15. The process of any preceding claim, wherein the stabilizing agent is selected from the group consisting of collagen, chondroitin sulphate, hydroxyapatite, crystalloids, organic solutions, molecules, elements and combinations thereof.
16. The process of any preceding claim, wherein the plurality of interactomes are selected from intracellular, intercellular, extracellular, transcellular, and pericellular interactomes, and combinations thereof.
17. The composition prepared by the process of any preceding claim.
18. A method, comprising administering the composition prepared by the process of any preceding claim.
19. The method of any preceding claim, wherein the composition prevents or reduces scarring upon administration.
20. A composition, comprising a stimulated acellular material selected from intracellular, intercellular, extracellular, transcellular, and pericellular interactomes, and combinations thereof derived from a triploblastic tissue interface.

### EXAMPLES

### Example 1

Harvest, extract, excise, remove, biopsy, punch, dissociate, digest, cleave, withdraw, isolate, part, or separate a form of composite integumental tissue from a system, material, substrate and/or tissue. Such action can occur through mechanical, chemical, enzymatic, electrical, biological and/or physical mechanism(s).

Place the composite integumental tissue in Solution A [an isotonic, biocompatible solution (e.g., 0.9% NaCl, HBSS, PBS, DMEM, RPMI, lactated ringers, 5% dextrose in water, 3.2% sodium citrate)+/−antimicrobial agent(s)] for 5 minutes and gently agitate, rock, shake, or stir.

Place the composite integumental tissue in Solution B [an isotonic, biocompatible solution (e.g., 0.9% NaCl, HBSS, PBS, DMEM, RPMI, lactated ringers, 5% dextrose in water, 3.2% sodium citrate)] for 5 minutes and gently agitate, rock, shake, or stir.

Place the composite integumental tissue in Solution A for 5 minutes and gently agitate, rock, shake, or stir.

Place the composite integumental tissue in Solution B for 5 minutes and gently agitate, rock, shake, or stir.

Remove the composite integumental tissue from Solution B and place in Solution C [an isotonic, biocompatible solution (e.g., 0.9% NaCl, HBSS, PBS, DMEM, RPMI, lactated ringers, 5% dextrose in water, 3.2% sodium citrate)] and locate an interface. Equipment and/or supportive systems may be used to locate the interface.

If a complete interface is not present, locate an area where a sub-compartment or sub-set of the interface is present or is likely present.

Harvest, extract, excise, remove, biopsy, punch, dissociate, digest, cleave, withdraw, isolate, part, or separate an interface compartment.

Obtain the acellular composition by:

a. mechanically, physically and/or energetically altering the interface through agitation, stress, shear and/or other forms of dematerialization;
b. chemically and/or electrically altering the ionic material; or
c. energetically disrupting the interface; and then isolating the acellular composition.

### Example 2

Harvest, extract, excise, remove, biopsy, punch, dissociate, digest, cleave, withdraw, isolate, part, or separate a form of composite integumental tissue from a system, material, substrate and/or tissue. Such action can occur through mechanical, chemical, enzymatic, electrical, biological and/or physical mechanism(s).

Place the composite integumental tissue in Solution A [an isotonic, biocompatible solution (e.g., 0.9% NaCl, HBSS, PBS, DMEM, RPMI, lactated ringers, 5% dextrose in water, 3.2% sodium citrate)+/−antimicrobial agent(s)] for 5 minutes and gently agitate, rock, shake, or stir.

Place the composite integumental tissue in Solution B [an isotonic, biocompatible solution (e.g., 0.9% NaCl, HBSS, PBS, DMEM, RPMI, lactated ringers, 5% dextrose in water, 3.2% sodium citrate)] for 5 minutes and gently agitate, rock, shake, or stir.

Place the composite integumental tissue in Solution A for 5 minutes and gently agitate, rock, shake, or stir.

Place the composite integumental tissue in Solution B for 5 minutes and gently agitate, rock, shake, or stir.

Remove the composite integumental tissue from Solution B and place in Solution C [an isotonic, biocompatible solution (e.g., 0.9% NaCl, HBSS, PBS, DMEM, RPMI, lactated ringers, 5% dextrose in water, 3.2% sodium citrate)] and locate an interface. Equipment and/or supportive systems may be used to locate the interface.

If a complete interface is not present, locate an area where a sub-compartment or sub-set of the interface is present or is likely present.

Harvest, extract, excise, remove, biopsy, punch, dissociate, digest, cleave, withdraw, isolate, part, or separate an interface compartment.

Obtain the acellular composition by:

d. mechanically, physically and/or energetically altering the interface through agitation, stress, shear and/or other forms of dematerialization;
e. chemically and/or electrically altering the ionic material; or
f. energetically disrupting the interface; and then isolating the acellular composition.

Formulate the composition.

Add the formulated composition to a biocompatible vector for storage, transport, preservation, use, deployment, or alteration. Alternatively, material(s) may also be place directly into living systems, partial living systems and/or synthetic supportive systems which permit the material(s) to persist and/or propagate.

Material(s) may be altered, changed, regulated, manipulated, adjusted, modified, transformed, converted, mutated, reconstructed, evolved, adapted, integrated and/or subtracted from and/or added to other material(s) directly and/or indirectly so as to change the primary material(s) in function, appearance, structure, makeup, behavior and/or existence within such system(s) and/or environment(s).

Deploy the formulated composition within targeted environment and/or system as necessary for function as primary product by utilizing a vector which may encompass one or a combination of: solid, semi-solid, liquid, semi-liquid, particle, fiber, scaffold, matrix, molecule, substrate, material, cellular entity, tissue entity, device, biologic, therapeutic, macromolecule, chemical, agent, organism, media and/or synthetic substance.

### Example 3: Preparation of Cutaneous-Derived Compositions

Cutaneous tissue specimens were removed from the dorsum of 12-week old Lewis rats and stored in chilled HBSS and subsequently rinsed for 5 minutes in a solution of HBSS and 0.1 mg/mL gentamicin in a sterile specimen cup.

In a laminar flow hood, tissues were individually removed from specimen containers and placed in a petri dish. HBSS+Dispase 5 U/μL was then added to each petri dish in a volumetric equivalent to the tissue specimen.

Specimen was then placed on a rocker for 6 hours at 37° C.+5% CO₂. Materials were then placed into a 50 cc conical tube. An additional volumetric equivalent of termination agent was added to the specimen.

An equal amount of RPMI was added to the material and placed on a rocker at 4° C. overnight.

After rocking, the mixture was subject to centrifugation at 10,000 rpm for 10 minutes resulting in a supernatant and a pellet of the remaining tissue debris. In a laminar flow hood, the supernatant from each cutaneous tissue specimen was removed, filtered with a with a 40 μm filter.

The filtrate was the added in a 1:1 ratio of a stock solution made from a base containing 800 mL of distilled water+10× [8 g of NaCl, 400 mg of KCl, 140 mg of CaCl2, 100 mg of MgSO4-7H2O, 100 mg MgCl2-6H2O, 60 mg of Na2HPO4, 60 mg of KH2PO4, 1 g of Glucose, and 350 mg of NaHCO3]. The combined solution was then placed into a centrifuge tube and stored at 4° C.

Semisolid materials (isolated from top portion after centrifugation) were removed from the tube and placed in molds for cryodesiccation. Molds were sprayed with silicone release spray prior to use. Freeze dryer settings included vacuum between 500-600 mTorr, 1.7° C./min ramp rate, freezing at −29° C. for 2 hours, primary drying at −18° C. for 40 hours, and secondary drying at 29° C. for 1 hour.

### Example 4: Raman Spectroscopy Experimental Conditions

A confocal Raman microscope (Thermo Fisher Raman DXR) with a 10× objective (N.A. 0.25) and a laser wavelength of 785 nm (28 mW of power at sampling point) was used to collect spectra. The estimated spot size on the sample was 2.1 μm and resolution was 2.3-4.3 cm-1. The confocal aperture used was a 25 μm slit, and spectra between wavenumbers 500-3500 cm-1 were collected. The Raman spectrum was recorded on a deep depletion charge-coupled device (CCD) detector. The recorded Raman spectrum was digitalized and displayed on a personal computer using OMNIC software. A total of 3-4 spectra were collected from 4 different points across the surface. Raman spectroscopy analysis was performed using OMNIC software for Dispersive Raman. Proprietary features available in OMNIC (Thermo Scientific) software were used to remove background fluorescence from all the spectra using polynomial baseline fitting (6th order) and to normalize the spectra. Spectra collected from different locations on a particular specimen were averaged to represent an individual specimen. Spectral data was collected using an exposure of 2 s with a signal to noise ratio of 300 to ensure specimen was homogeneous and the collected spectra represented the bulk material. Representative Raman shift spectroscopy data for different compositions disclosed herein can be found below.

### Example 5: Characterization of Compositions Prepared from Chondral-Derived Materials

Compositions prepared as disclosed herein from chondral-derived materials were characterized by Raman spectroscopy. FIG. 2 shows the average Raman spectrum of a solution composition and the average Raman spectrum of a cryodesiccated composition.

### Example 6: Characterization of Compositions Prepared from Osseous-Derived Materials

Compositions prepared as disclosed herein from osseous-derived materials were characterized by Raman spectroscopy. FIG. 3 shows the average Raman spectrum of the compositions: the average Raman spectrum of the solution material (top), the average Raman spectrum of the cryodesiccated material (middle), and the average Raman spectrum of the gel material (bottom).

### Example 7: Characterization of Compositions Prepared from Musculoskeletal-Derived Materials

Compositions prepared as disclosed herein from musculoskeletal-derived materials were characterized by Raman spectroscopy. FIG. 4 shows the average Raman spectrum of the compositions: solution composition (top), cryodesiccated composition (middle), and gel composition (bottom).

### Example 8: Characterization of the Compositions Prepared from Cancellous Osseous-Derived Materials

A composition prepared as disclosed herein from cancellous osseous-derived materials was characterized by Raman spectroscopy. FIG. 5 shows the average Raman spectrum of the gel composition.

### Example 9: Characterization of Compositions Prepared from Myo-Derived Materials

Compositions prepared as disclosed herein from myo-derived materials were characterized by Raman spectroscopy. FIG. 6 shows the average Raman spectra of: a solution composition (top), a cryodesiccated composition (middle), and a gel composition (bottom).

### Example 10: Characterization of Compositions Prepared from Tendon

A composition prepared as disclosed herein from tendinous-derived materials was characterized by Raman spectroscopy. FIG. 7 shows the average Raman spectrum of the gel composition.

### Example 11: Characterization of Compositions Prepared from Osseous Trabecula-Derived Materials

Compositions prepared as disclosed herein from osseous trabecula-derived materials were characterized by Raman spectroscopy. FIG. 8 shows the average Raman spectra of: a gel composition (top), a cryodesiccated composition (middle), and a solution composition (bottom).

### Example 12: Rheological Experimental Conditions

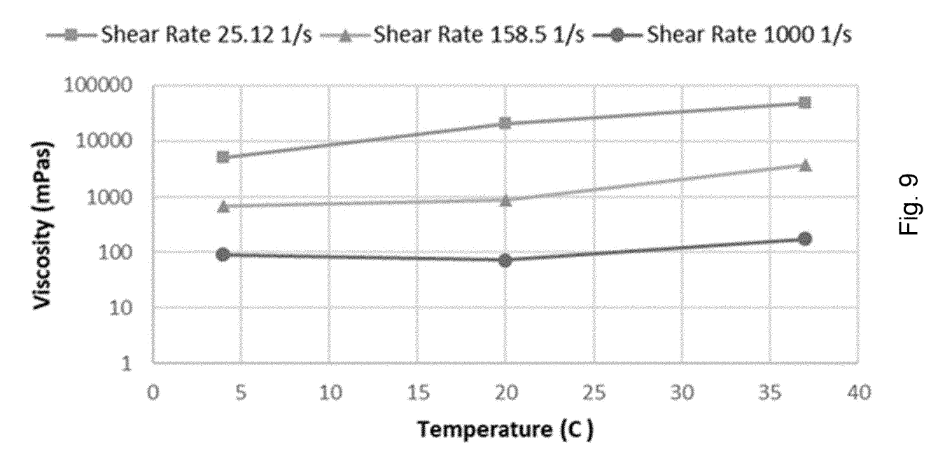

In FIG. 9, a HAAKE Modular Advanced Rheological System fitted with a 35 mm diameter plate geometry and Peltier plate temperature control system from Thermo Scientific was used to determine rheological properties of gel. Viscosity test consisted of a shear rate step test from 1-1000 1/s with 16 steps distributed logarithmically. In FIG. 9, the gel was removed from 4° C. and placed at room temperature (20° C.) and in a water bath (37° C.). After four days, the rheology test was performed. The 4° C. sample was tested immediately after removal from 4° C. refrigerator.

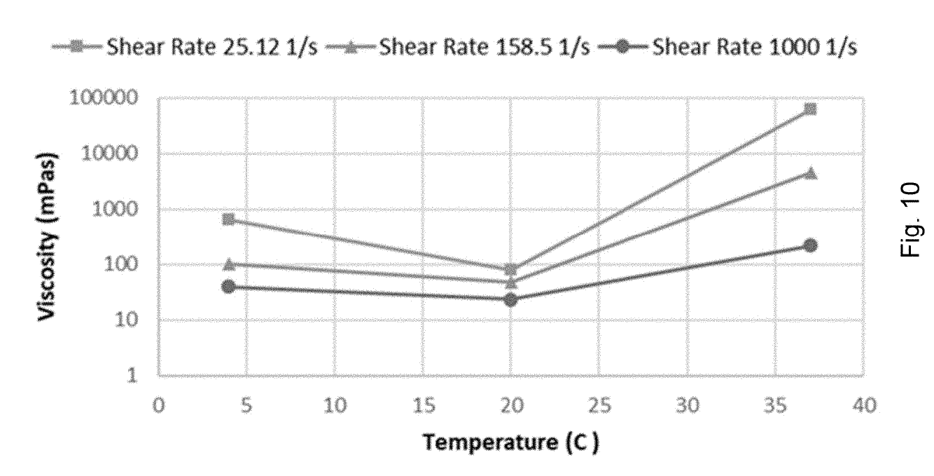

In FIG. 10, the gel was removed from 4° C. and placed at room temperature (20° C.) and in a water bath (37° C.). After four days, the rheology test was performed. The 4° C. sample was tested immediately after removal from 4° C. refrigerator.

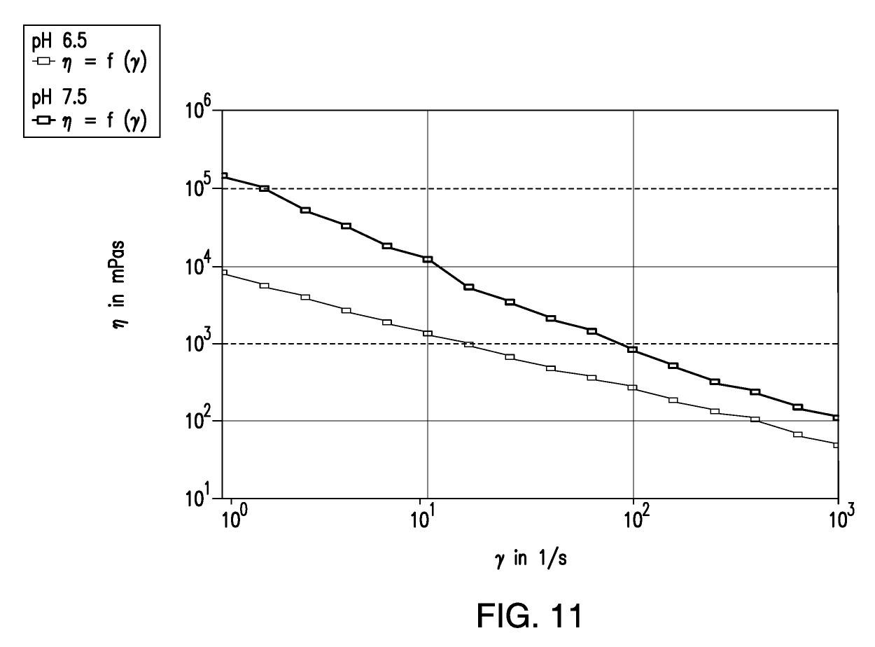

In FIG. 11, the gel was removed from 4° C. and set out at room temperature (20° C.) for one hour. After warming at room temperature, two samples were tested on the rheometer. The first sample was an initial pH of 6.5. The second sample was adjusted to pH 7.5 using 1M NaOH. The rheology test consisted of a shear rate step from 1-1000 1/s with 16 steps distributed logarithmically.

### Example 13: Characterization of Compositions Using SEM (Scanning Electron Microscopy) and Instron Universal Testing Machine (UTM)

The scaffold internal architecture and microstructure were examined by scanning electron microscopy (SEM), EVO 10 LS Environmental Scanning Electron Microscope (Carl Zeiss Microscopy LLC, NY) fitted with an electron back scatter detector was used. Scaffolds were tested in compression using an electronic UTM with 1 kN load capacity (Instron, MA, USA) at a constant crosshead velocity of 0.5 mm/min until crushing failure occurred. The compressive load and displacement were recorded at 0.1 s intervals during testing. Five samples were tested for each type of scaffold in order to determine mean modulus of elasticity.

### Example 14: Preparation of Freeze-Dried, Gel, and Solution Compositions

For each of long bone (rabbit), long bone with surrounding muscle (rabbit and mouse), and muscle (rabbit and mouse), a freeze-dried composition, a gel composition, and a solution composition were prepared. Materials and methods for each preparation are described below.

Methods:

Tissue was cleaned in the following order: 1^{st} wash, 1^{st} rinse, 2^{nd} wash, 2^{nd} rinse. The washes consisted of 5-minute agitation in saline with 0.01% (w/v) gentamicin. The rinses consisted of 5-minute agitation in saline. After cleaning, the tissue was processed by disrupting a tissue interface to create a stimulated composition comprising an aggregate of living core potent cellular entities and supportive entities where the living core potent cellular entities express a sequence of LGR4, LGR5, and/or LGR6. Processed tissue was placed in 50 mL conical tubes with a 1:1 10×HBSS to tissue volume ratio. Tissue and HBSS were rocked for 36-48 hours at 4° C. then centrifuged at 5000 rpm for 15 minutes. Supernatant was removed, strained through a 40 μm mesh, and placed in molds for lyophilization. Molds were sprayed with silicone release spray prior to use. Freeze dryer settings included vacuum between 500-600 mTorr, 1.7° C./min ramp rate, freezing at −29° C. for 2 hours, primary drying at −18° C. for 40 hours, and secondary drying at 29° C. for 1 hour.

Dialysis:

1. Filter the composition through #40 size mesh.
2. Load the composition into dialysis tubing (Spectra/Por Dialysis Membrane MWCO: 100-500 D, Spectrum Labs 131057)
3. Place loaded dialysis tubing in an appropriate buffer of desired osmolarity using 1:100 sample to buffer volume in fridge on shaker. For example:
a. 5×HBSS for 2-3 hours, followed by 1×HBSS for 4-5 hours, followed by 1×HBSS overnight
4. Remove sample from dialysis tubing and collect in a conical tube and centrifuge at 1200 g and 4° C. for 20 minutes
6. Remove supernatant from solution to yield the same volume as in step 2.b

Rinse:

Rinsing was performed according to the following protocol:

1. Filter the composition through #40 size mesh (Cell dissociation sieve, Sigma CD1-1KT)
2. Load the composition into desired mold
3. Freeze dry samples (24-hour profile, Labconco freeze dryer)
4. Remove samples from mold
5. Rinse samples in saline using details below:
a. Each rinse consisting of 1 mL of saline for every 5 mg of sample
b. Total of 5 rinses (replace with new saline for each rinse) for 10 minutes per rinse

### Example 15: Compressive Modulus

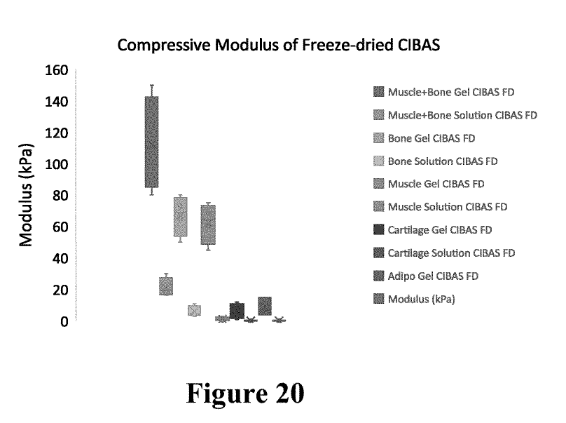

Freeze-dried compositions as prepared in Example 14 were tested for compressive (modulus) strength using an electronic UTM (Universal testing machine) with 1 kN load capacity (Instron, MA, USA) at a constant crosshead velocity of 1 mm/min until break point was reached. N=2 samples were tested for every type. The load and displacement values were recorded at 0.1 s intervals during testing. FIG. 20 shows compressive modulus of rabbit muscle and bone freeze-dried compositions.

### Example 16: Protein Analysis

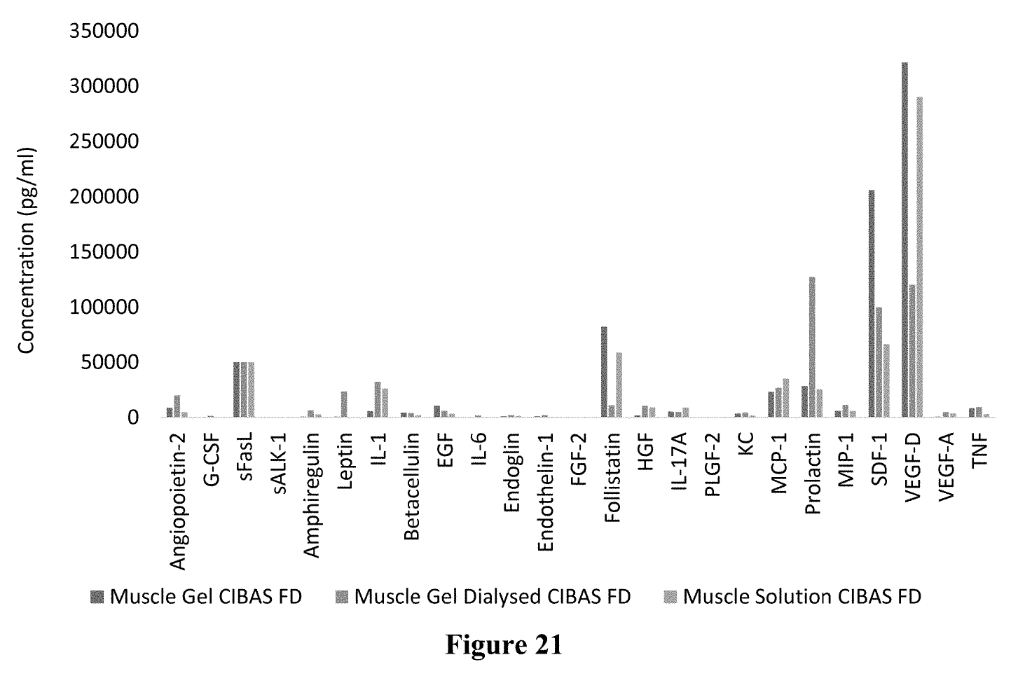

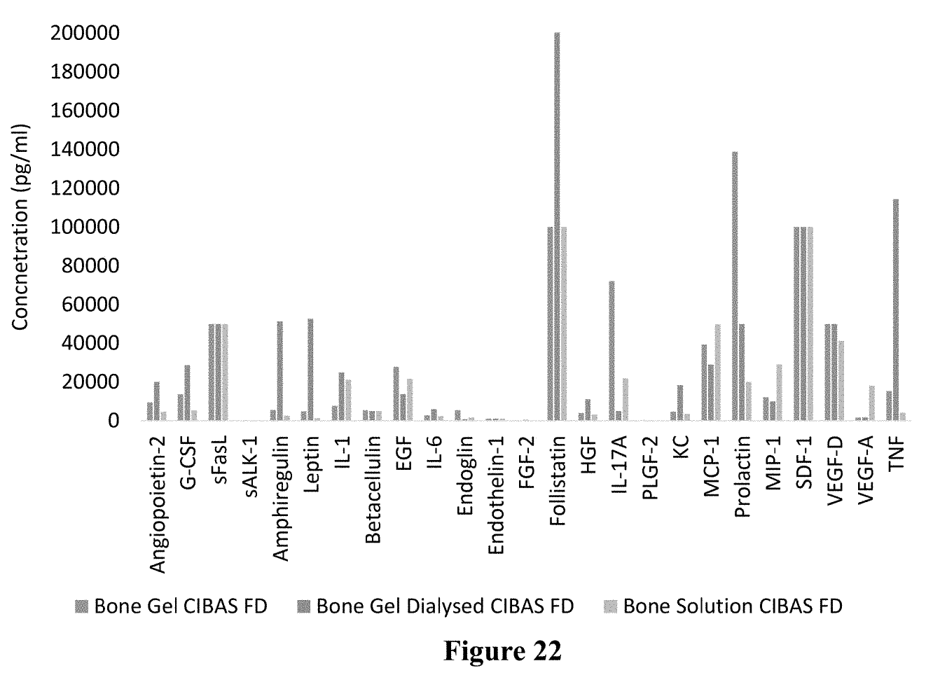

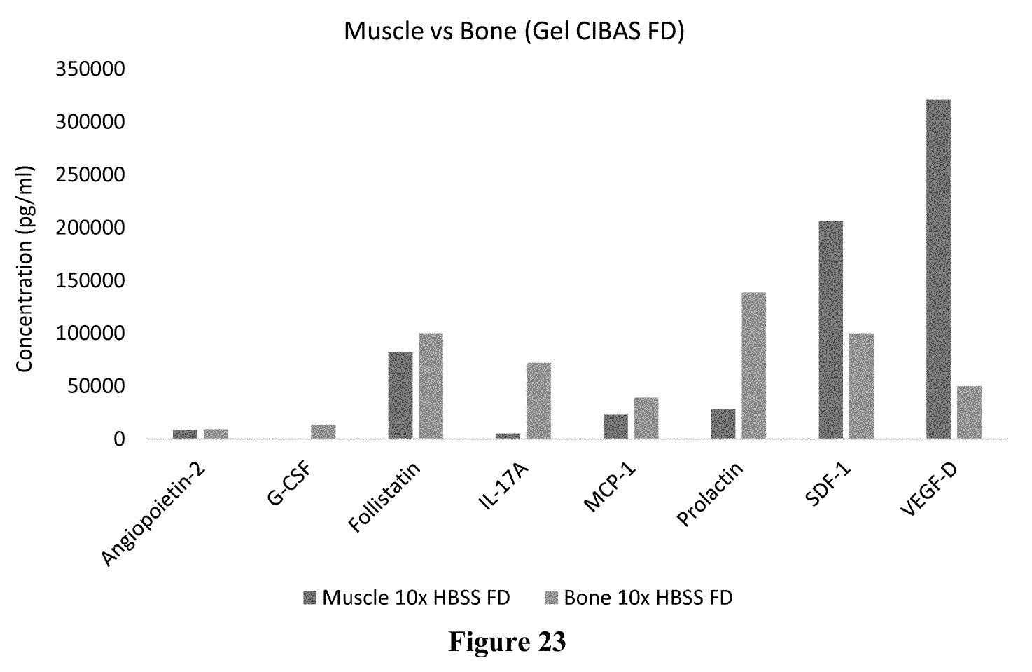

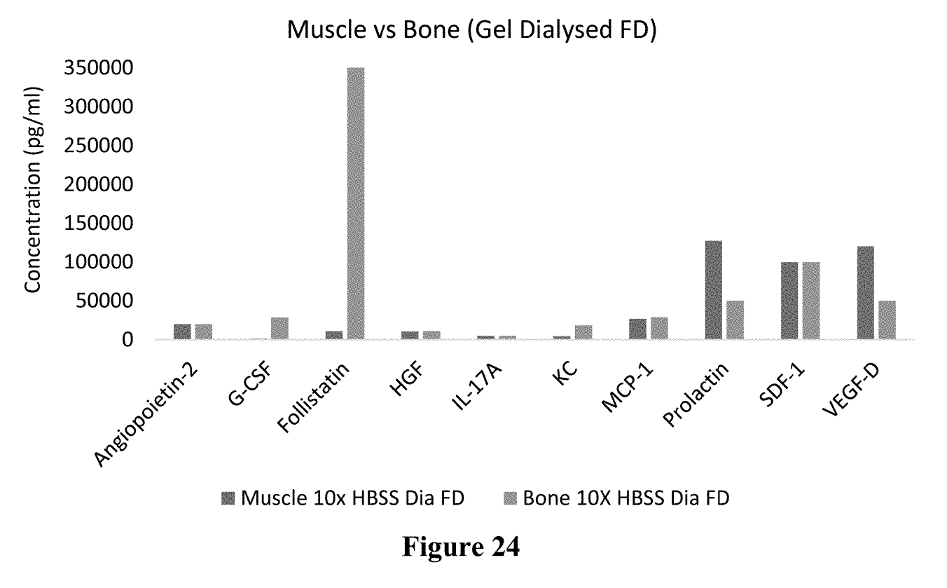

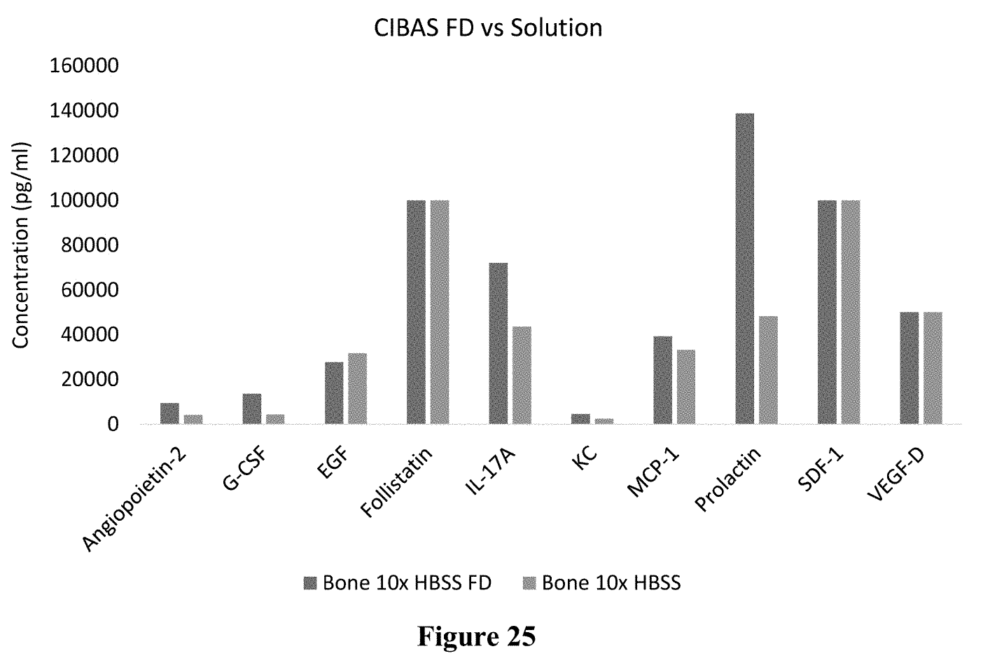

MILLIPLEX® MAP Mouse Angiogenesis/Growth Factor Magnetic Bead Panel was used as an assay for proteins for muscle and bone compositions prepared in Example 14. In particular, MAGPMAG-24K, a 24-plex (for serum/plasma) kit, was used for the simultaneous quantification of the following analytes: Angiopoietin-2, granulocyte-colony stimulating factor (G-CSF), sFasL, sAlk-1, Amphiregulin, Leptin, IL-1b, Betacellulin, EGF, IL-6, Endoglin, Endothelin-1, FGF-2, Follistatin, HGF, PECAM-1, IL-17a, PLGF-2, KC, monocyte chemoattractant protein-1 (MCP-1), Prolactin, MIP-1a, stromal cell derived factor (SDF-1), VEGF-C, VEGF-D, VEGF-A, and tumor necrosis factor (TNF). FIGS. 21-25 show the results of the protein assay.

### Example 17: Biomarker Analysis

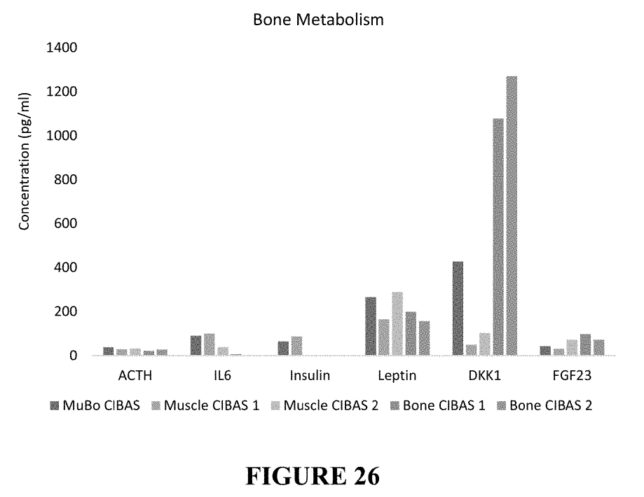

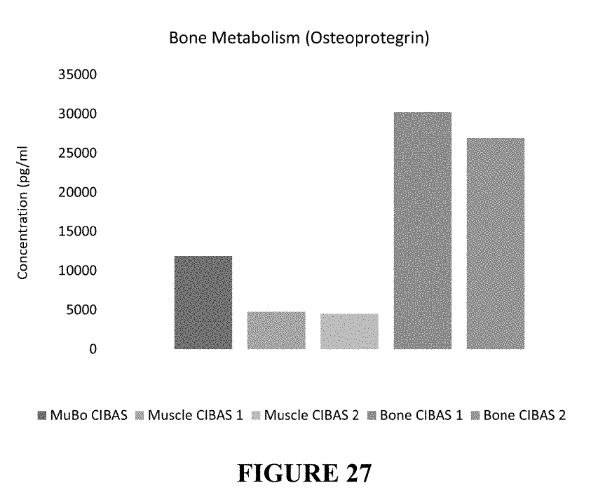

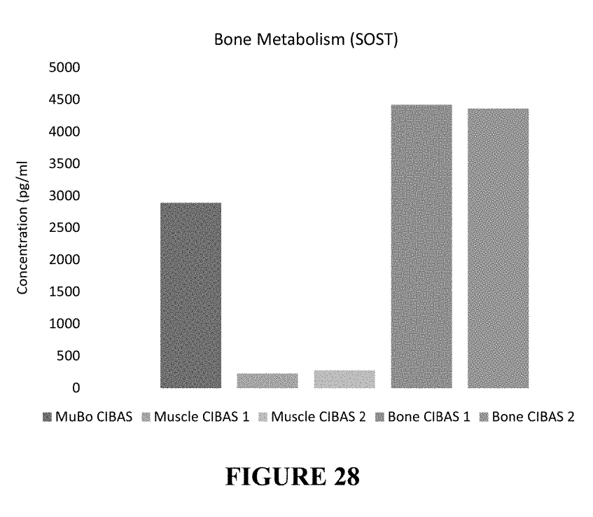

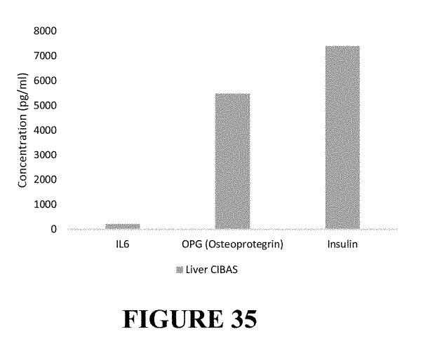

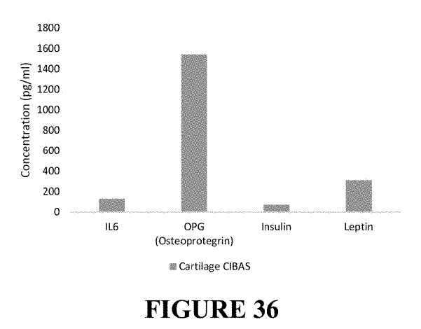

MILLIPLEX® MAP Mouse Bone Magnetic Bead Panel—Bone Metabolism Multiplex Assay was used for characterization of muscle and bone compositions prepared in Example 14. The Milliplex® MAP Mouse Bone Magnetic Bead Panel contains all the components necessary to measure the following in any combination: ACTH (Adrenocorticotropic hormone), DKK-1 (Dickkopf WNT Signaling Pathway Inhibitor 1), IL-6, Insulin, Leptin, TNFα, OPG (Osteopotegrin), SOST and FGF-23. FIGS. 26-28 show the results of this assay. FIGS. 35 and 36 also show the results of this assay for a liver-derived composition and a cartilage-derived composition, respectively.

### Example 18: Comparative Raman Spectroscopy Analysis

Method:

Tissue was cleaned in the following order: 1^{st} wash, 1^{st} rinse, 2^{nd} wash, 2^{nd} rinse. The washes consisted of 5-minute agitation in saline with 0.01% (w/v) gentamicin. The rinses consisted of 5-minute agitation in saline. After cleaning, the tissue was processed by disrupting a tissue interface to create a stimulated composition comprising an aggregate of living core potent cellular entities and supportive entities where the living core potent cellular entities express a sequence of LGR4, LGR5, and/or LGR6. Processed tissue was placed in 50 mL conical tubes with a 1:1 saline to tissue volume ratio. Tissue and saline were rocked for 36-48 hours at 4° C. then centrifuged at 5000 rpm for 15 minutes. Supernatant was removed, strained through a 100 μm mesh, and stored at −20 C for analysis. Raman spectroscopy analysis was performed in accordance with Example 4 comparing the compositions to native tissue specimen.

Results:

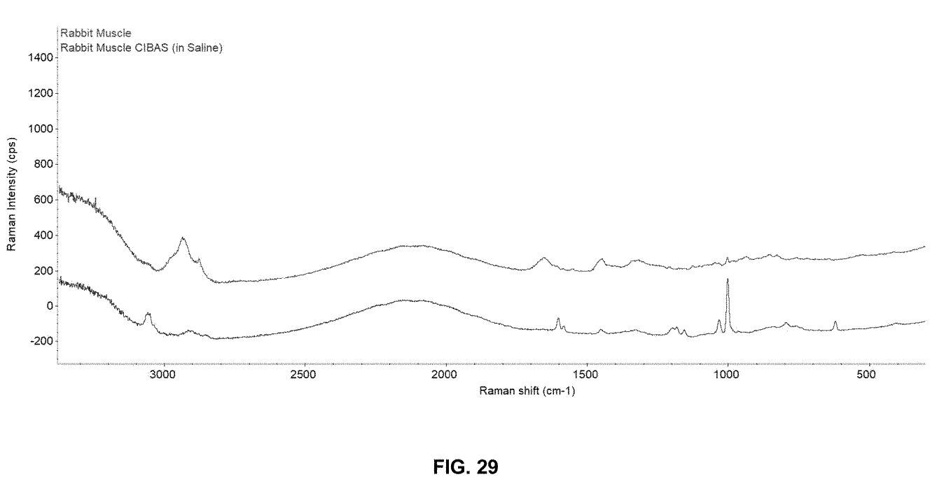

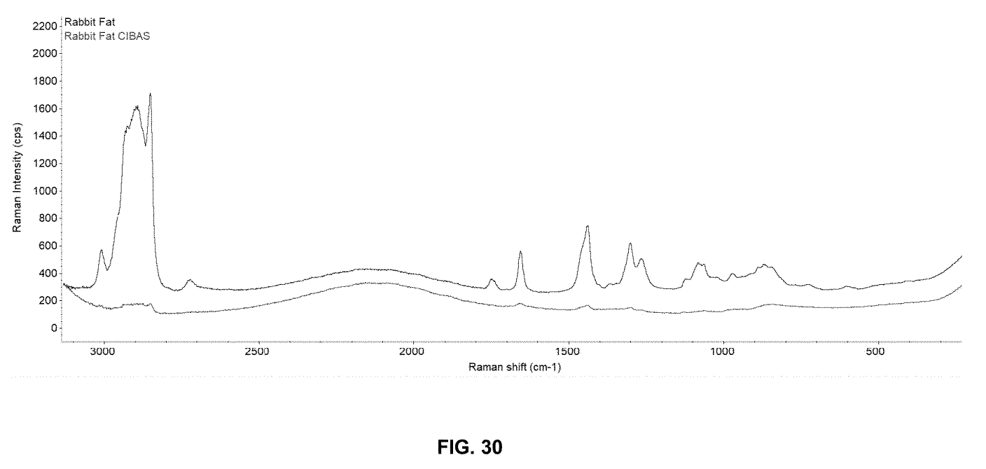

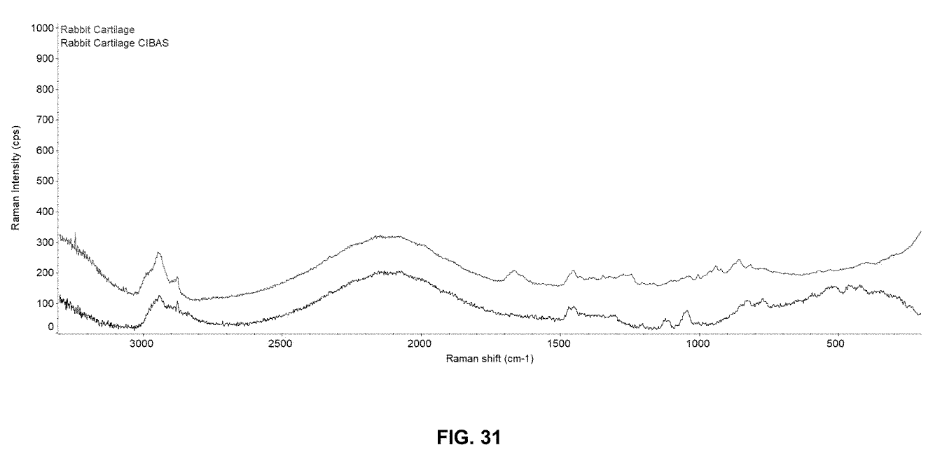

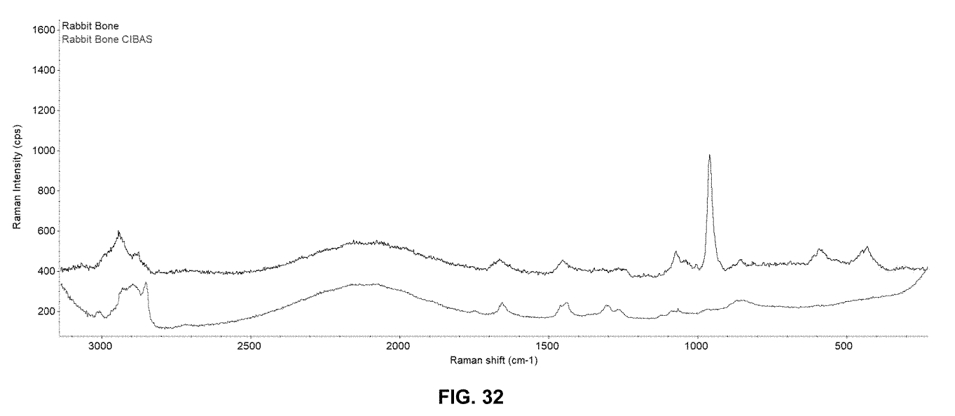

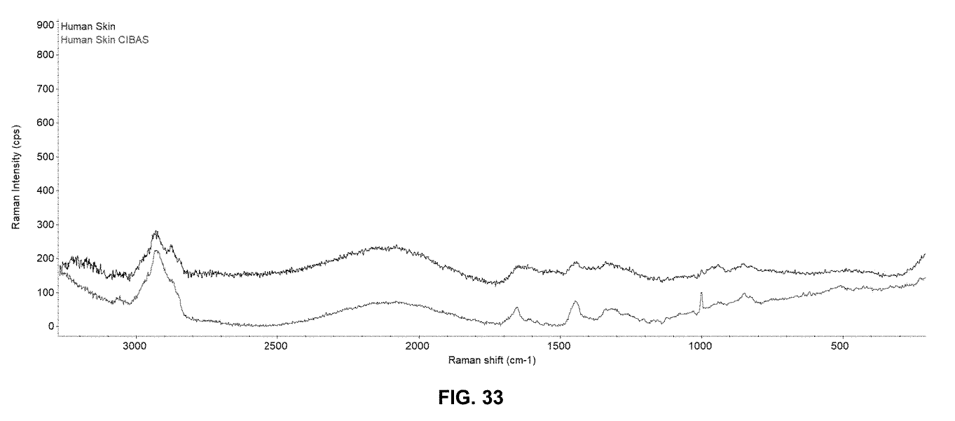

FIGS. 29-33 show the results of the comparative Raman spectroscopy analysis and the corresponding differences between the molecular fingerprints of the compositions versus the respective native tissue specimens from which the compositions were derived. FIG. 29 shows the Raman spectrum of a rabbit muscle-derived composition (bottom) providing an altered molecular fingerprint compared to that of native rabbit muscle (top). FIG. 30 shows the Raman spectrum of a rabbit fat-derived composition (bottom) providing an altered molecular fingerprint compared to that of native rabbit fat (top). FIG. 31 shows the Raman spectrum of a rabbit cartilage-derived composition (bottom) providing an altered molecular fingerprint compared to that of native rabbit cartilage (top). FIG. 32 shows the Raman spectrum of a rabbit bone-derived composition (bottom) providing an altered molecular fingerprint compared to that of native rabbit bone (top). FIG. 33 shows the Raman spectrum of a human skin-derived composition (bottom) providing an altered molecular fingerprint compared to that of native human skin (top).

### Example 19: Preparation of Muscle-Derived Composition

Harvest rabbit thigh muscle using sharp dissection. Tissue is rinsed in deionized water for 3 cycles, followed by rinsing with an isotonic solution (e.g. 0.9% NaCl). Dissociate tissue and disrupt the cellular and non-cellular interfaces by placing 10 grams of tissue into a 50 cc conical tube (Conical A) and combining with a 40 mL collagenase/trypsin solution (0.2% trypsin, 0.2% collagenase type IV, 50 μg/ml gentamycin in 50 ml of DMEM/F12). Gently agitate combination for 30 minutes at 37° C. Combine with volumetric equivalent of termination agent. Centrifuge solution at 1000 RPM for 10 minutes and transfer supernatant to a 50 cc conical (Conical B). Re-suspend contents in Conical A in 10 mL DMEM/F12 with 40 μL of DNase (2 U/μL) and incubate at room temperature for 5 minutes with occasional agitation. Centrifuge at 1000 RPM for 5 minutes and transfer supernatant to Conical B. Rinse contents of Conical A with 10 mL DMEM/F12 and agitate for 120 minutes at room temperature. Centrifuge at 100 RPM for 2 minutes. Transfer composite integumental tissue and supernatant to Conical B. Add 20 mL 0.9% NaCl to Conical A and incubate at 4° C. for future combination and/or further dissociation of intercellular compartments. Incubate Conical B at 4° C. until the addition of the contents of Conical A. Thereafter, incubate Conical B for 120 minutes at room temperature followed by overnight incubation on a rocker at 4° C. Resultant composition should have a pH within a range of 4.8 to 8.5 and osmolarity of 199 and 800 mOsm/Kg. Semisolids and supernatant are transferred to open face containers coated with silicone release spray of a desired surface area and height and filled to desired thickness. Product can be preserved or solidified using cryodesiccation using freeze dryer settings including a vacuum between 500-600 mTorr, 1.0° C./min ramp rate, freezing at −35° C. for 3 hours, and primary drying at −20° C. for 45 hours. Resultant composition can be stored or upon need, combined with a biocompatible compound such as 0.9% NaCl, HMS, DMEM/F12, or RPMI to create physical characteristics and viscosity required of application.

### Example 20: Preparation of Muscle/Osseous-Derived Composition

Harvest rabbit thigh muscle en bloc with segment of associated osseous tissue using sharp dissection and transfer to an adequately sized vessel. Tissues are submerged in deionized water for 5 minutes. Solution is decanted and process is repeated for a total of 3 cycles. Tissues are submerged in an isotonic solution with 0.01% (w/v) gentamicin for 5 minutes. Tissues are then combined with biocompatible solution with a concentration range of 1×-10× (i.e. 1×-10×NaCl) in a ratio of 0.5:1 to 1:10 (v/v) and mechanically dissociated with resulting particulate sizes of 5 mm³ to 1 cm³. Add EDTA to a concentration of 10 mM to 0.5M and incubate on a rocker at 4° C. overnight. Resulting composition is centrifuged at 1000 RPM for 15 minutes and remaining tissues are removed from solution. Remaining disrupted cellular interfaces are combined 1:1 volume to 10×HBSS and incubated on a rocker for 2 hours at room temperature and then stored overnight at 4° C. Solution is centrifuged at 100 RPM for 5 minutes. Composite integumental tissue and supernatant are transferred to open face silicone ready release coated containers of desired size and surface area. Compositions are heat desiccated at 37° C. for 48 hours. Following desiccation, samples can be frozen at −20° C. for storage or gently combined with 0.9% NaCl and incubated for 2 hours at 4° C. and centrifuged at 100 RPM for 5 minutes and supernatant is discarded.

### Example 21: Preparation of Adipose-Derived Composition

Subcutaneous, visceral, and/or brown rabbit adipose tissue is collected and placed in a 50 cc conical tube and submerged in an isotonic solution with 0.01% (w/v) gentamicin at 4° C. for 10 minutes. Tissues are then transferred to a 50 cc conical tube and combined with an isotonic solution (e.g. 1×HBSS, 0.9% NaCl, or 1×DMEM) and shaken vigorously for 5 minutes at 4° C. Composition is centrifuged at 500 RPM for 2 minutes, supernatant is discarded, and cycle is repeated 2 additional times. Composition is combined 1:1 (v/v) with 10×DMEM and incubated on a rocker for 2 hours at room temperature. Composition is transferred to a 50 cc conical tube and passed through a 100 μM filter three times and centrifuged at 900 g for 15 minutes. Oil separates are removed and remaining disassociated interfaces and supernatant are transferred to a 50 cc conical and incubated overnight at 4° C. Additional passive oil separates are removed. Consistency of composition can be further stiffened by cross-linking with additional treatments including calcium chloride or glutaraldehyde.

### Example 22: Preparation of Adipose-Derived Composition

Subcutaneous, visceral, and/or brown rabbit adipose tissue is collected and placed in a 50 cc conical tube and submerged in an isotonic solution with 0.01% (w/v) gentamicin at 4° C. for 10 minutes. Tissues are then transferred to a 50 cc conical tube and combined with an isotonic solution (e.g. 1×HBSS, 0.9% NaCl, or 1×DMEM) and shaken vigorously for 5 minutes at 4° C. Composition is centrifuged at 500 RPM for 2 minutes, supernatant is discarded, and cycle is repeated 2 additional times. Composition is combined with DMEM and 0.1% collagenase for 1 hour at 37° C. followed by dispase 5 U/μL for two hours at 37° C. Composition is combined with a volumetric equivalent of termination agent. Tissues are centrifuged at 2000 RPM for 10 minutes. Oil/adipose layer is removed and remaining cellular interfacing and dissociated material is combined with 0.5:1 (v/v) 10×HBSS for 2 hours at room temperature on a rocker. Tissues are vortexed at 600 VPM and combined with 1:1 (v/v) 5×HBSS and rocked for 2 hours at 4° C. Tissues are vortexed at 600 VPM and combined with 1:1 (v/v) 1×HBSS and rocked overnight at 4° C. Composite integumental tissue and supernatant are transferred to open face silicone ready release coated containers of desired size and surface area. Compositions are heat desiccated at 25° C. for 4 hours followed by curing at 37° C. for 40 hours. Following desiccation, samples can be frozen at −20° C. for storage or gently combined with 0.9% NaCl and incubated for 2 hours at 4° C. and centrifuged at 100 RPM for 5 minutes and supernatant is discarded.

### Example 23: Cell Viability Experiment

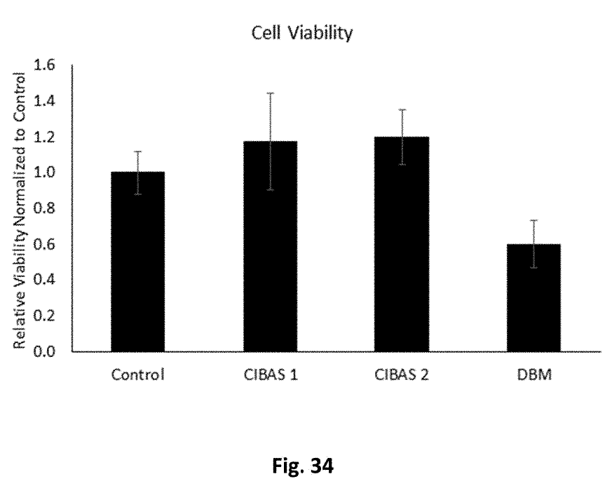

Human osteosarcoma cells (MG-63) alone (control) or co-cultured with various osseous tissue-derived compositions or a commercially available human-derived demineralized bone matrix (DBM) were evaluated for viability/proliferation using the Alamar blue assay. Cells co-cultured with the tissue-derived compositions demonstrated increased viability as compared to control cells showing that the compositions disclosed herein increased cellular proliferation and viability as shown in FIG. 34. Accordingly, FIG. 34 demonstrates compositions as disclosed herein include stimulated biological material and augment the generation or healing of native tissue.

Methods:

Cell Preparation:

1. MG-63 Cells (passage P+5) were thawed in complete DMEM (10% FBS, 50 μg/ml Gentamicin) media and plated in a 75 cm² flask until confluent (˜1 week). Cells were trypsinized and moved to 4 new 75 cm² flasks and grown to confluence, then trypsinized again and moved to 20 new flasks. The confluent flasks were trypsinized, resuspended in 18 ml of freezing medium (90% fetal bovine serum, 10% DMSO) and frozen at −80° C. in a Nalgene Cryol C Freeing Container (Cat#5100-001). The cell vial label reads:

### MG-63 Cell Line (Human Osteosarcoma)

### Sigma Cat#86051601; Lot#14K002

### Passage 8

2. Residual cells were placed in 3 flasks and grown to ˜90% confluence for the viability experiment.
3. The scaffold plugs were placed in 48-well plates and rehydrated in 500 μl complete DMEM for 1 hour (note: column 6 was filled with 500 μl media only and served as a scaffold-free control).
4. MG-63 cells were trypsinized and resuspended in media. A total of 0.5×10⁵ cells per well (125 μl volume) were added to each well of rows D-F. An additional 125 μl of complete DMEM was added to wells in row C to act as a cell-free control. Cells were incubated overnight at 37° C., 5% CO₂.

Measuring cytotoxicity or proliferation using alamarBlue by spectrophotometry:

1. Cells were harvested which were in the log phase of growth and cell count was determined. Cell count was adjusted to 1×10⁴ cells/ml.
2. Cells were plated and combined with reagents to be tested.
3. Mixing by shaking ensued and then alamarBlue was aseptically added in an amount equal to 10% of the volume in the well.
4. Cultures were incubated with alamarBlue for 4-8 hours. N.B.
5. Cytotoxicity or proliferation was measured using spectrophotometry of fluorescence.
6. Absorbance was measured at wavelengths of 570 nm and 600 nm after incubation. A blank media only was used.
7. Percent difference in reduction between treated and control cells in cytotoxicity and proliferation assays was calculated by:

Percentage difference between treated and control cells=[(O2×A1)−(O1×A2)/(O2×P1)−(O1×P2)]×100

From the foregoing detailed description, it will be evident that modifications and variations can be made to the methods and compositions disclosed herein without departing from the spirit or scope of the disclosure. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A composition comprising stimulated biological material derived from an interface compartment, wherein the composition is capable of augmenting the generation or healing of a native tissue when administered to a subject in need thereof.

2. The composition of claim 1, wherein the stimulated biological material derived from the interface compartment is acellular.

3. The composition of claim 1, wherein the stimulated biological material comprises biological material derived from a heterogeneous population of mammalian tissue interface cells, wherein the heterogeneous population of mammalian tissue interface cells are associated with a plurality of interactomes.

4. The composition of claim 3, wherein the stimulated biological material includes living core potent cellular entities and supportive entities.

5. The composition of claim 4, wherein the living core potent cellular entities express RNA transcripts and/or polypeptides of one or more of LGR4, LGR5, LGR6, Pax 7, Pax 3, MyoD, Myf 5, keratin 15, keratin 5, cluster of differentiation 34 (CD34), Sox9, c-Kit+, Sca-1+ or any combination thereof.

6. The composition of claim 4, wherein the supportive entities comprise cellular populations, extracellular matrix elements, or any combination thereof.

7. The composition of claim 6, wherein the extracellular matrix elements comprise one or more of hyaluronic acid, elastin, collagen, fibronectin, laminin, extracellular vesicles, enzymes, and glycoproteins.

8. The composition of claim 1, wherein the stimulated biological material is derived from a triploblastic tissue interface, an osseous tissue interface, a cutaneous tissue interface, a musculoskeletal tissue interface, an adipose tissue interface, or a cartilage tissue interface.

9. The composition of claim 1, wherein the composition further comprises an agent selected from the group consisting of a pharmaceutical, an enzyme, a molecule, and any combination thereof.

10. A kit comprising the composition of claim 1 and instructions for use.

11. A method for augmenting tissue regeneration or healing of native tissue a subject in need thereof comprising administering to the subject an effective amount of the composition of claim 8.

12. The method of claim 11, wherein the native tissue is skin and administration of the composition prevents or reduces scarring in the subject.

13. A method for preparing the composition of claim 1 comprising
stimulating at least a portion of a mammalian interface compartment of a tissue specimen to generate stimulated biological material, wherein the mammalian interface compartment comprises a heterogeneous population of mammalian tissue interface cells; and
isolating a fraction of the stimulated biological material, and
optionally wherein the stimulating occurs in the presence of a biocompatible material selected from the group consisting of a pharmaceutical agent, an enzyme, a molecule, and combinations thereof.

14. The method of claim 13, wherein the portion of the mammalian interface compartment is stimulated using mechanical stimulation, chemical stimulation, enzymatic stimulation, energetic stimulation, electrical stimulation, biological stimulation, or any combination thereof.

15. The method of claim 13, wherein the fraction of the stimulated biological material is an acellular fraction.

16. The method of claim 13, further comprising adding a biocompatible transfer agent to the stimulated biological material, wherein the biocompatible transfer agent is selected from alginate, gelatin, petroleum, collagen, mineral oil, hyaluronic acid, crystalloid, chondroitin sulfate, elastin, sodium alginate, silicone, PCL/ethanol, lecithin, a poloxamer, and any combination thereof.

17. The method of claim 13, further comprising preserving the isolated fraction of the stimulated biological material via dessication or cryodessication.

18. The method of claim 13, further comprising adding a stabilizing agent to the isolated fraction of the stimulated biological material.

19. The method of claim 13, further comprising incubating the stimulated portion of the mammalian interface compartment for about 12 to 72 hours prior to isolating the stimulated biological material.

20. The method of claim 13, wherein the fraction of the stimulated biological material is isolated by centrifugation, filtration, or a combination thereof.
